(19) United States
(12) Patent Application Publication
(10) Pub. No.: US 2023/0077464 A1
(43) Pub. Date: Mar. 16, 2023

(54) WEARABLE ELECTRONIC DEVICES, SYSTEMS, AND METHODS FOR COLLECTING PATIENT MOTION DATA AND ASSESSING PATIENT ACTIVITY

(71) Applicant: Veracron LLC, Ypsilanti, MI (US)

(72) Inventors: Grant Kruger, Ypsilanti, MI (US); Steven Harte, Livonia, MI (US)

(73) Assignee: Veracron LLC, Ypsilanti, MI (US)

(21) Appl. No.: 18/050,985

(22) Filed: Oct. 29, 2022

Related U.S. Application Data
(63) Continuation of application No. PCT/US2021/030183, filed on Apr. 30, 2021.
(60) Provisional application No. 63/018,102, filed on Apr. 30, 2020.

(51) Int. Cl. A61B 5/11; A61B 5/00

(52) U.S. Cl. CPC A61B 5/1118; A61B 5/681; A61B 5/4848; A61B 5/0002; A61B 2562/0219; A61B 2562/0257; A61B 2560/0242; A61B 2560/0214; A61B 2560/029; A61B 2560/0475

(57) ABSTRACT

A wearable electronic device includes a light sensor configured to sense environmental light, a timer that provides time indicators, a motion sensing unit that senses motion and outputs motion data according thereto, and a data storage that receives and stores the motion data or other data derived therefrom in association with the time indicators. After sensing environmental light with the light sensor, the timer begins providing the time indicators and the motion sensing unit begins sensing the motion.

### CROSS-REFERENCE TO RELATED APPLICATION(S)

This application is a continuation of International PCT Application No. PCT/US2021/030183, filed Apr. 30, 2021, which claims priority to and the benefit of U.S. Provisional Application No. 63/018,012, filed Apr. 30, 2020, the entire disclosures of which are incorporated by reference herein.

### TECHNICAL FIELD

This disclosure relates to assessing patient activity and, specifically, assessing patient activity objectively with motion data of the patient.

### BACKGROUND

Physicians often prescribe pain interventions to patients for pain management, for example, to manage pain associated with chronic conditions or after medical procedures. To assess the efficacy of the pain interventions, physicians are typically reliant on the patient providing feedback. However, patient feedback is subjective and, therefore, may be an unreliable and/or inaccurate indicator of the efficacy of the pain interventions.

### SUMMARY

Disclosed herein are implementations of wearable electronic devices, systems, and methods for assessing patient activity.

In one implementation, a wearable electronic device is provided for collecting motion data of patients for assessing activity thereof. The wearable electronic device generally includes a motion sensing unit, a data storage, a communications interface, a power source, and an electronics housing. The motion sensing unit senses motion and outputs motion data according thereto. The data storage receives and stores the motion data. The communications interface is for transferring the motion data from the data storage. The electronics housing is configured to be worn by a patient. To one or more of transfer the motion data from the data storage or physically access the power source, the electronics housing must be permanently deformed.

In one implementation, a wearable electronic device is provided for collecting motion data of a patient. The wearable electronic device generally includes a motion sensing unit, a data storage, a communications interface, a controller, and a power source. The motion sensing unit includes one or more sensors for sensing motion and outputting motion data according thereto. The data storage receives and stores the motion data. The communications interface is for transferring the motion data from the data storage. The controller operates the motion sensing unit and the data storage. The wearable electronic device does not include any output device by which the patient can directly observe an output of the wearable electronic device, and does not include an input device by which the patient can directly provide an intentional input to the wearable electronic device.

In one implementation, a method is provided for assessing activity of multiple patients. The method includes: (1) distributing one or more wearable electronic devices of a plurality of the wearable electronic devices to each of one or more patients to be worn thereby; (2) collecting, with each of the wearable electronic devices of the plurality, motion data of the patient while the wearable electronic device is being worn; (3) receiving, at a processing facility, each of the wearable electronic devices of the plurality from the patients; and (4) transferring, with a computer data system associated with the processing facility, the motion data from each of the wearable electronic devices of the plurality.

In one implementation, a wearable electronic device includes a light sensor configured to sense environmental light, a timer that provides time indicators, a motion sensing unit that senses motion and outputs motion data according thereto, and a data storage that receives and stores the motion data or other data derived therefrom in association with the time indicators. After sensing environmental light with the light sensor, the timer begins providing the time indicators and the motion sensing unit begins sensing the motion.

After sensing the light with the light sensor, the timer may continue providing the time indicators until subsequent data transfer between the data storage and a computing device. The wearable electronic device may further include a proximity sensor, and after sensing environmental light with the light sensor, the proximity sensor may begin operating to sense a patient wearing the wearable electronic device. After sensing the patient wearing the wearable electronic device, the motion sensing unit may begin sensing the motion. In response to sensing environmental light with the light sensor, the proximity sensor may begin operating to sense a patient wearing the wearable electronic device. In response to sensing the patient wearing the wearable electronic device, the motion sensing unit may begin sensing the motion. In response to both environmental light not being sensed by the light sensor and motion not being sensed by the motion sensing unit, recording of the motion data may stopped. The wearable electronic device may include a power source, and after sensing environmental light with the light sensor, the power source may begin providing power to the motion sensing unit. After sensing environmental light with the light sensor, the power source may begin providing power to the proximity sensor. The wearable electronic device may include a controller that includes the timer. The wearable electronic device may include proximity sensor, and the controller may begin the motion sensing unit to begin sensing the motion upon sensing a patient wearing the wearable electronic device with the proximity sensor. The wearable electronic device may include a light source that outputs light at 10 lumens or less. The wearable electronic device may include a communications interface by which the motion data may be transferred from the data storage to a computing device. The data storage may store the other data that is the root mean square of motion data from each of three axes of the motion sensing unit. The wearable electronic device may include a housing and a flexible circuit to which the light sensor, the timer, the motion sensing unit, the data storage, and the proximity sensor are coupled. The flexible circuit may include two outer portions that form electrodes of the proximity sensor, the proximity sensor may be a capacitive sensor, and/or the flexible circuit may be positioned within the housing with the two outer portions cooperatively extending around a majority of a periphery of an inner surface of the housing. The housing may be cylindrical. The housing may environmental light to pass therethrough to the light sensor.

In one implementation, a system may include the wearable electronic device and packaging in which the wearable electronic device is positioned, the packaging being opaque and preventing light from reaching the light sensor. Upon removing the wearable electronic device from the packaging, the light sensor senses environmental light after which the timer may begin providing the time indicators and the motion sensing unit begins sensing the motion.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1A is a side elevation view of a wearable electronic device with hidden components depicted in broken lines.

FIG. 1B is a top view of the wearable electronic device of FIG. 1A.

FIG. 1C is a cross-sectional view of the wearable electronic device of FIG. 1A in a first state and taken along line 1C-1C in FIG. 1A.

FIG. 1D is a cross-sectional view of the wearable electronic device of FIG. 1A in a second state and taken along line 1D-1D in FIG. 1A.

FIG. 1E is a side view of another embodiment of a wearable electronic device.

FIG. 2A is a schematic view of the wearable electronic device of FIG. 1A.

FIG. 2B is a schematic view of another embodiment of a wearable electronic device.

FIG. 2C is a schematic view of a flexible circuit board of the wearable electronic device of FIG. 2B.

FIG. 2D is a cross-sectional view of the wearable electronic device of FIG. 1E taken along line 2D-2D and including the flexible circuit board of FIG. 2C.

FIG. 3 is a schematic of an example controller of the wearable electronic device of FIG. 1A.

FIG. 4A is a top view of the wearable electronic device of FIG. 1A in packaging.

FIG. 4B is a cross-sectional view of the wearable electronic device of FIG. 1A with a removable cover and taken along line 1C-1C.

FIG. 5 is a flowchart of a first method for collecting motion data of a patient.

FIG. 6 is a schematic of a system for assessing activity of multiple patients.

FIG. 7 is a flowchart of a method for assessing activity of multiple patients.

### DETAILED DESCRIPTION

Disclosed herein are devices, systems, and methods for collecting physiological data that, among other uses, may provide an objective indicator, used alone or in conjunction with other indicators, to assess efficacy of pain interventions. In particular, it is theorized that patient activity, which may be assessed from motion data measured with an accelerometer, may indicate efficacy of the pain interventions, for example, with higher levels of activity indicating greater efficacy of the pain interventions.

In one implementation, the devices may be configured as wearable electronic devices that collect motion data and distributed to patients as prescribed to be worn for a prescribed wear period of time. The devices may be configured as limited-time use (e.g., one-time use) devices that are worn by a patient for a limited period of time and which may be constrained to such usage times and patient uses by power capacity, data storage capacity, and/or limitations on the manners in which the patient may interact with the device (e.g., patients are unable to transfer or otherwise access motion data). The patient sends the device to a processing facility that processes the device to transfer and assess the collected motion data and from which an activity assessment is generated and sent to the prescriber. The processing facility may, in some implementations, also restore electronics of the device for reuse in the same or another device. The processing facilities may be centrally- or regionally-located to ensure short transit times.

As discussed in further detail below, the wearable electronic device may be configured to avoid influencing behavior of the patient and may be configured to present few and/or low barriers to use (e.g., few user instructions, no electronic interaction, and/or no additional equipment) by the patient and the prescriber (e.g., a physician). To avoid influencing behavior of the patient, the wearable electronic device may be configured, for example, to receive few or no patient inputs, provide few or no patient outputs, and be physically compact and lightweight. To present few or low barriers to use, the wearable electronic device may be configured, for example, to require little or no maintenance by the patient or the prescriber (e.g., by requiring no charging, no electronic interaction for updates, and/or being provided as a single-use device) and to require little or no electronic interaction by the patient or prescriber (e.g., by requiring no electronic equipment to start recording, transfer, or process the motion data).

As also discussed in further detail below, a system and method are provided for collecting the motion data from one or more patients with one or more of the wearable electronic devices. The system and method are configured to present few and/or low barriers to use by the patient and the prescriber, while also providing a low-cost system for collecting the motion data from and assessing the activity of multiple different patients, such as many thousands of patients. In addition to those aspects described previously, various electronic components of the wearable electronic device may be configured for reuse, while a system and method may include processing facilities that process the wearable electronic devices to assess the motion data and, in some implementations, may manufacture new single-use devices from the electronics of other ones of the wearable electronic devices.

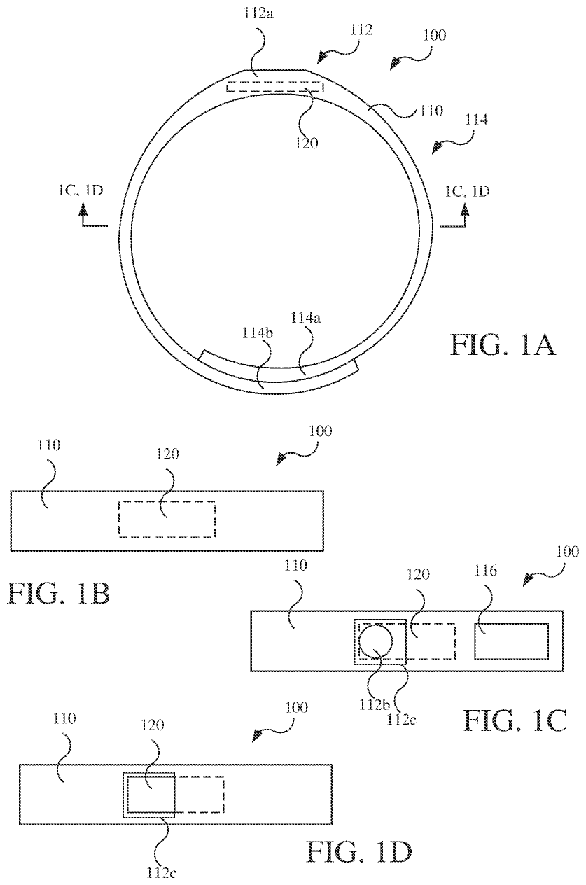

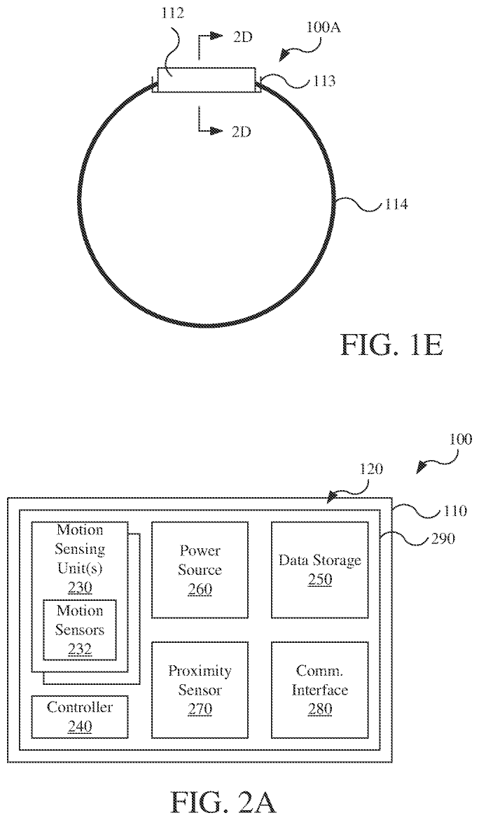

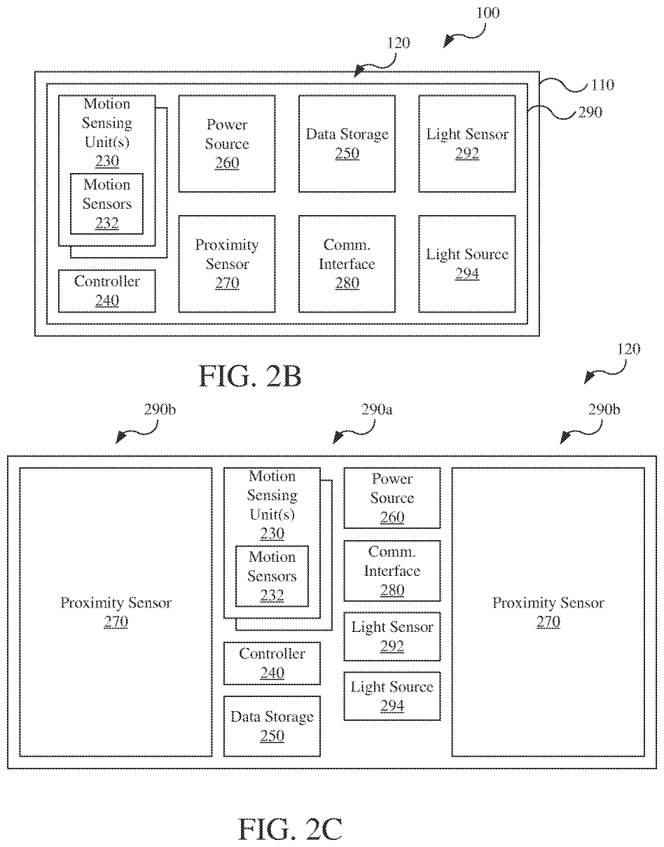

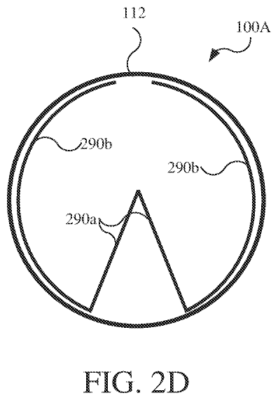

Referring to FIGS. 1A to 2, a wearable electronic device 100 generally includes a body 110 and electronics 120 coupled to the body 110. The body 110 is configured to be worn by the patient, such as on a wrist of the patient, and generally includes a housing portion 112 and a coupling portion 114. The electronics 120 are configured to collect physiological data of the patient, such as movement data. As shown schematically in FIG. 2A, the electronics 120, for example, include one or more motion sensing units 230, a controller 240, a data storage 250, a power source 260, and a communications interface 280, which may be coupled to a substrate 290 and form an electronics module. The electronics 120 may, in some embodiments, also include other components, such as a proximity sensor 270.

The body 110 is configured to be worn by the patient, such as on a wrist of the patient, such that the electronics 120 move with the patient for sensing motion of the patient. The body 110 generally includes a housing portion 112 and a coupling portion 114.

The housing portion 112 is coupled to the electronics 120. For example, the housing portion 112 may include or form an electronics housing 112a that defines a chamber that contains the electronics 120 and that may further be sealed to prevent water from reaching the electronics 120. The electronics housing 112a may be have a greater thickness and/or a greater width than the coupling portion 114 to accommodate the electronics 120 disposed therein.

The wearable electronic device 100 may be configured as a wrist-worn device (e.g., a band), in which case the coupling portion 114 is elongated and extends from the housing portion 112 around the wrist of the patient. As shown, the body 110 may be adjustable in length with the coupling portion 114 having two band segments 114a, 114b that extend from either side of the housing portion 112 and are coupleable to each other (e.g., with a clasp or other suitable coupling mechanism). Alternatively, the coupling portion 114 may be continuous and adjust only elastically to the size of the wrist of the patient.

In still further alternatives, the wearable electronic device 100 may be configured to couple to the user in other manners in which case the coupling portion 114 may be configured as a hook or clip (e.g., to attach to clothing of the user) or an adhesive (e.g., to attach to the skin or clothing of the user).

The body 110 is configured to conform to the patient. The body 110 may be both flexible and elastic, so as to hold the electronics 120 in close proximity to the user and to provide comfort to the user. For example, the housing portion 112 may include a layer of a flexible and/or elastic (e.g., compressible) material arranged between the electronics 120 (e.g., a module thereof) and the patient, such as an elastomeric material (e.g., silicone), and may conform to the patient by bending around the patient and/or by compressing. The two band segments 114a, 114b of the coupling portion 114 may be formed of a flexible and/or elastic material, such as an elastomeric material (e.g., silicone), which may conform to the patient by bending around the patient and/or by elastically extending (e.g., as the patient moves their wrist). The housing portion 112, including the electronics housing 112a, and the coupling portion 114 (e.g., the two band segments 114a, 114b) may be formed monolithically with each other (e.g., of the same polymeric compound, such as an elastomer (e.g. silicone). Alternatively, the electronics housing 112a may be formed of a different material (e.g., plastic), which may be further surrounded (e.g., encapsulated) by an elastomeric material of the housing portion 112 that may be formed monolithically with the coupling portion 114.

The body 110 and, in particular, the housing portion 112, may be configured in different manners for different functions associated with the electronics 120, such as operation of the power source 260, providing physical access for retrieving the motion data from the data storage 250 and/or accessing the power source 260, and/or removal of the electronics 120 for repurposing in another wearable electronic device 100. In each case, the electronics 120 may be sealed within the electronics housing 112a of the housing portion 112 to protect the electronics 120 from water.

Referring to FIG. 1C, the housing portion 112 may be configured to permit the flow of air to the power source 260, which may be configured as a metal-air battery that requires air for operating (e.g., to support a reduction reaction). In such case, the housing portion 112 includes a membrane 112b that is water-impermeable and seals the cavity formed by the electronics housing 112a, but which is air-permeable.

The membrane 112b may be formed in different manners. The membrane 112b may be visibly distinguishable from other portions of the body 110 or may be indistinguishable. The membrane 112b may be provided on an inner surface adjacent the patient (as shown) or any other location.

In one example, the membrane 112b is formed monolithically with the electronics housing 112a of the housing portion 112, such as with an elastomer (e.g., silicone) in a molding operation that forms that housing portion 112 (i.e., both the electronics housing 112a and the membrane 112b) and, thereby, encases the electronics 120. In such case, the housing portion 112 may itself form the membrane 112b. In another example, the membrane 112b is formed separately from and is coupled to the electronics housing 112a, such as with a sheet material or other component (e.g., formed of polytetrafluoroethylene) that is coupled to the electronics housing 112a (e.g., being molded therein or coupled with an adhesive thereto). In another example, the membrane 112b is formed to the electronics housing 112a, for example, as a silicone material that is applied and cured to electronics housing 112a to form the membrane 112b and seal the cavity thereof.

In other examples, the power source 260 may be another type of battery or a capacitor, which does not require air for operation thereof. In such cases, the membrane 112b is not required, although the electronics housing 112a may still be formed of an air-permeable material (e.g., silicone).

The housing portion 112 may, instead of or in addition to being configured to permit air flow to the power source 260, provide for physical access to the electronics 120, such as the power source 260 and/or the communications interface 280. Referring additionally to FIG. 1D, in such case, the housing portion 112 may include a removable segment 112c that, when removed, opens an opening 112d that provides physical access to the electronics 120 and, in particular, the power source 260 and/or the communications interface 280 (not separately shown in FIG. 1D). Physical access to the power source 260 may allow for replacement thereof (e.g., in the case of the power source 260 being a primary battery) or recharging thereof (e.g., in the case of the power source 260 being a secondary battery). Physical access to the communications interface 280 may allow for physical connection thereto with a physical (e.g., conductive) connection, such as with a proprietary or standardized interface.

The removable segment 112c may be configured in various different manners. In one example, the wearable electronic device 100 is a reusable device, while the removable segment 112c is sacrificial, so as to not be capable of resealing the opening 112d of the housing portion 112. In one example, the removable segment 112c may be formed of silicone that is formed with the electronics housing 112a or is applied and cured to the electronics housing 112a. The removable segment 112c is removed by being permanently deformed, for example, by being torn from the housing portion 112. By requiring permanent deformation for removal of the removable segment 112c, the wearable electronic device 100 may be considered a single-use device. The removable segment 112c and the housing portion 112 may be cooperatively configured for removal of the removable segment 112c without damaging the housing portion 112, for example, with the removable segment 112c being formed of a weaker and/or thinner material and/or with an intermediate member arranged therebetween (e.g., a more rigid material component defining the opening 112d). In another example, the removable segment 112c may be a separate member (e.g., a removable cover) that is coupled to the housing portion 112, such as with an adhesive (e.g., a sticker) or a cap (e.g., coupled mechanically to the housing portion 112, such as with a press-fit, threaded, and/or clipped). The removable segment 112c may form the membrane 112b.

The housing portion 112 may, instead of or in addition to being configured to permit air flow to the power source 260 and/or provide physical access to the electronics 120, be configured for removal of the electronics 120 from the body 110. The wearable electronic device 100 may, for example, be configured as a single-use device that is to be worn by the patient for a limited time frame. The body 110 may be a sacrificial component, while the electronics 120 are provided as a reusable module that may subsequently be incorporated into another wearable electronic device 100 with a new one of the bodies 110. The body 110 may be formed, for example, by molding a polymer compound, such as an elastomer (e.g., silicone) or plastic (e.g., ABS plastic), to form the housing portion 112 around the electronics 120. The body 110 is configured for removal of the electronics 120 in a process that requires permanent deformation (e.g., irreversibly damaging) the housing portion 112 of the body 110, such as by cutting and/or tearing the material forming the housing portion 112 of the body 110. The body 110 and the electronics 120 may be cooperatively configured to facilitate removal in a repeatable manner of the electronics 120 from the bodies 110 of different ones of the wearable electronic devices 100 and/or without damage to the electronics 120 being removed. For example, housing portion 112 may be formed of a weaker and/or thinner material than surrounding material or may include an isolated weakened region (e.g., a thinned region). Instead or additionally, the electronics 120 may include a mechanical feature that facilitates cutting and/or tearing of the housing portion 112 of the band, such as a pointed edge (e.g., formed by a circuit board thereof).

The body 110 may further include markings 116 that may be machine and/or human readable. The markings 116 may, for example, include a unique identifier, such as a serial number having alphanumeric characters that are readable by a human and/or bar code that is readable by a machine. The human-readable identifier may allow the prescriber to associate a particular one of the wearable electronic devices 100 with a particular patient (e.g., in a medical health record), which may be printed on or formed in the material forming the body 110 or on printed label affixed thereto. The machine-readable identifier allows a system, such as at the processing facility, to identify the particular one of the wearable electronic devices 100 for processing operations. The markings 116 may further include machine-readable orientation indicators, which allow a system, such as at the processing facility, to locate and orient the wearable electronic devices 100 for subsequent processing (e.g., for data transfer, restoration, disassembly, and/or recycling).

The various features of the body 110 described previously may be provided in any suitable combination. In a first preferred example, the body 110 includes a singular, monolithic polymeric component that forms the housing portion 112 with the electronics 120 being entirely sealed by the housing portion 112 (e.g., being molded therein). The coupling portion 114 may also be formed monolithically with the housing portion 112. In the first preferred example, the body 110 may be configured as a single-use component that is configured to require permanent deformation of the body 110 for accessing the communications interface 280 or otherwise accessing or removing the electronics 120. In the first referred example, the polymer of the housing portion 112 may form the membrane 112b. The polymer compound may be an elastomer, such as silicone, or plastic, such as ABS plastic.

In a second preferred example, the body 110 includes a singular, monolithic polymeric component that forms the housing portion 112, along with the membrane 112b that is formed separately from and coupled to the housing portion 112 to seal the electronics 120 therein (e.g., being molded therein). The coupling portion 114 may also be formed monolithically with the housing portion 112. In the second preferred example, the body 110 may be configured as a single-use component that is configured to require permanent deformation of the body 110 for accessing the communications interface 280 or otherwise accessing or removing the electronics 120. The polymer may be an elastomer, such as silicone, or plastic, such as ABS plastic.

In a third preferred example, the body 110 includes a singular, monolithic polymeric component that that forms the electronics housing 112a of the housing portion 112 and the coupling portion 114, while the electronics housing 112a includes the opening 112d that is sealed with a removable segment 112c to seal the electronics 120 within the housing portion 112. In the third preferred example, the body 110 may be a multi-use component (e.g., to be worn by different patients). The removable segment 112c may be formed in any of the manners described above. The polymer may be an elastomer, such as silicone, or plastic, such as ABS plastic.

Referring to FIG. 1E, a wearable electronic device 100A is a variation of the wearable electronic device 100 and generally includes a housing portion 112 and a coupling portion 114, which are separately formed and coupled to each other. The housing portion 112 may, as shown, be configured as a housing (e.g., a container) that contains the electronics 120, while the coupling portion 114 is configured as a band that is coupled to the housing portion 112. The housing portion 112 may, for example, be configured as a canister that is sealed (e.g., being substantially cylindrical) or a container having another suitable shape (e.g., rectilinear or other box having a lid ultrasonically welded).

The coupling portion 114 is configured as a band (e.g., a strap), which may be sufficiently elastic to be stretched over the hand of the user to hold the housing portion 112 in close proximity to the wrist of the user, or be inextensible (e.g., insufficiently to stretch over the hand of the user) and releasably coupleable to the housing portion 112 so as to be receivable around the wrist of the user. The housing portion 112 may include a female component 113 (e.g., hooks) on ends thereof that receive and couple to ends of the coupling portion 114 (e.g., ends of the band). The female component 113 may, for example, may be smaller than a nominal dimension (e.g., diameter) of the coupling portion 114, so as to receive and compress the coupling portion 114 therein and, thereby, retain the coupling portion therein. The housing portion 112 may be made with any suitable material according to any suitable manufacturing method, such as polyethylene terephthalate glycol (PETG) or other polymer via injection molding, extrusion, and/or additive manufacturing. The housing portion 112 may be sealed in any suitable manner with the electronic 120 therein, for example, by ultrasonic welding or otherwise sealingly coupling covers or caps (not illustrated) thereto.

Referring to FIG. 2A, as referenced above, the electronics 120 include one or more of the motion sensing units 230, the controller 240, the data storage 250, the power source 260, and the communications interface 280, which may be coupled to the substrate 290 to form an electronics module. In some embodiments, the electronics 120 may also include a proximity sensor 270. As described above, the electronics 120 are coupled to the body 110, for example, being provided as a module that is encapsulated by the body 110 (e.g., being sealed in the housing portion 112 thereof). Furthermore, as discussed below, the electronics 120, or the module thereof, may be configured to conform to the patient when wearing the wearable electronic device 100.

The motion sensing unit 230 is configured to sense motion of the patient, such as motion of the wrist of the patient on which the wearable electronic device 100 is worn, and outputs motion data according thereto. The motion sensing unit 230 may sense the motion while the wearable electronic device 100 is operated in a motion data collection mode. As will be discussed in further detail below, the motion sensing unit 230 may begin sensing motion upon activation of the power source 260 (e.g., when a metal-air battery is exposed to air), upon detection of environmental light (e.g., with a light sensor 292, discussed in further detail below), and/or or upon detection of the wearable electronic device being worn (e.g., detecting the patient with the proximity sensor 270).

The motion sensing unit 230 includes one or more sensors that sense motion, which may be referred to as motion sensors 232. The term motion is considered to include acceleration and velocity, such as linear acceleration, linear velocity, rotational acceleration, and rotational velocity. Motion may be measured directly, for example, with motion sensors that measure linear acceleration (i.e., accelerometers) or rotational velocity (i.e., gyroscopes). Alternatively, motion may be calculated from other measurements, such as linear velocity or cumulative displacement being derived from measured linear acceleration or linear acceleration and/or linear velocity being derived from measured position.

As discussed in further detail below, the motion sensing unit 230 may be configured to measure different motion, such as linear acceleration and/or rotational velocity, and environmental conditions that may influence the motion measurements. However, for the wearable electronic device 100 to be compact, lightweight, and low cost, it may be particular advantageous for the wearable electronic device 100 (i.e., the motion sensing unit 230) to perform only one or few types of motion measurement (e.g., linear acceleration in one or more axes), while not performing other types of motion measurement (e.g., rotational velocity) or position measurements from which motion can be calculated (e.g., local or global positioning). As a result, the number and cumulative size of the motion sensing unit 230 and the motion sensors thereof, by performing only one type of motion measurement, may be lower than if performing additional types of motion or position measurement, and the power consumption and associated power source may also be reduced.

The motion sensing unit 230, or the motion sensors thereof, may be configured to measure motion at any frequency suitable for assessing activity of the patient. For example, the motion sensors may measure motion at a frequency of between 1 Hz and 60 Hz, such as between 10 Hz and 30 Hz (e.g., 10 Hz), more or less. Further, the motion sensing unit 230 may be configured to measure motion for sensing time periods at regular sensing time intervals (e.g., epochs), such as for time periods between 2 seconds and 30 seconds at sensing time intervals between 30 seconds and 10 minutes (e.g., for 10 seconds every 1 minute).

Further, the motion sensing unit 230, or the motion sensors thereof, may be considered to include additional features or components suitable for outputting the measured motion as motion data, such as power management, analog filter(s), analog-to-digital converter(s), digital filter(s), control logic, and/or an input/output (I/O).

In a first preferred example, the motion sensing unit 230 includes one or more motion sensors that measure linear acceleration in one or more axes and may be referred to as accelerometers. For example, the motion sensing unit 230 measures linear acceleration in three axes, for example, being or including a three-axis accelerometer. Alternatively, the motion sensing unit 230 may measure acceleration in fewer than three axes, such as one or two axes. The accelerometer may, for example, be a micro-electromechanical system (MEMS) three-axis accelerometer, which may be provided as a singular device (e.g., a chip) or multiple devices (e.g., separate chips that each measure acceleration in one axis).

In the first preferred example, the motion sensing unit 230 may also include one or more additional sensors that measure one or more environmental conditions that influence measurements of the motion sensors and/or which may have other purposes. Such an additional sensor may be referred to as an environmental sensor. The environmental sensor of the motion sensing unit 230 may be or include a temperature sensor. In one specific example, the motion sensing unit 230 is or includes a combined accelerometer, such as a three-axis accelerometer, and a temperature sensor that are provided cooperatively as a singular device (e.g., a chip).

In the first preferred example, the wearable electronic device 100 and the motion sensing unit 230 thereof measure motion only as linear acceleration and are configured to not measure other motion or determine motion in another manner without measuring linear acceleration. That is, the wearable electronic device 100 and the motion sensing unit 230 include motion sensors that consist only of the accelerometers but not other types of motion sensors (e.g., gyroscope) or position sensors (e.g., local or global positioning sensors) from which motion data could be derived.

In a second preferred example, in addition to measuring linear acceleration, the motion sensing unit 230 includes one or more motion sensors that measure rotational velocity about one or more axes and may be referred to as gyroscopes. In one specific example, the motion sensing unit 230 measures rotational velocity about three axes, for example, being or including a three-axis gyroscope. Alternatively, the motion sensing unit 230 may measure rotational velocity about fewer than three axes, such as one or two axes. The gyroscope may, for example, be a micro-electromechanical system (MEMS) three-axis gyroscope, which may be provided as a singular device with the accelerometer (e.g., an inertial measurement unit, such as a singular chip), as a singular device in addition to the accelerometer (e.g., another chip), or multiple devices (e.g., separate chips that each measure rotational velocity about one axis).

In the second preferred example, the motion sensing unit 230 may include the environmental sensor, such the temperature sensor, as described above.

In the second preferred example, the wearable electronic device 100 and the motion sensing unit 230 thereof measure linear acceleration and rotational velocity and are configured to not measure other motion or determine motion in any other manner without measuring linear acceleration or rotational velocity. That is, the wearable electronic device 100 and the motion sensing unit 230 includes motion sensors that consist only of the accelerometers and the gyroscopes but not other types of motion sensors or position sensors (e.g., local or global positioning sensors) from which motion data could be derived.

In still further examples, the wearable electronic device 100 and the motion sensing unit 230 may include the accelerometer, the gyroscope, and/or other sensors that directly measure motion or position from which motion may be derived.

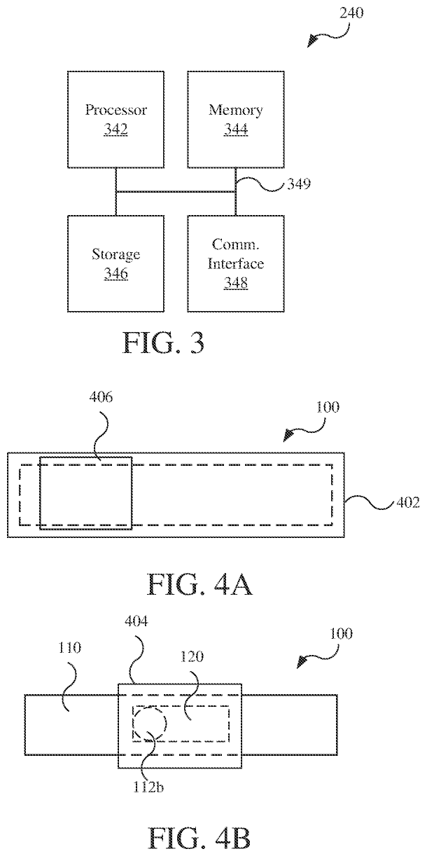

Referring to FIG. 3, the controller 240 is generally configured to control one or more operations of the electronics 120 of the wearable electronic device 100. For example, the controller 240 may operate the motion sensing unit 230 or collect the motion data upon activation of the power source 260, upon detection of the patient with the proximity sensor 270, and/or upon detection of some other criteria (e.g., measured motion meeting a threshold criterion, detection of light exceeding a threshold criterion with a light sensor (e.g., an ambient light sensor, such as the light sensor 292 discussed in further detail below), or upon a time criterion).

In one non-limiting example, the controller 240 generally includes a processing unit 342, a memory 344, a storage 346, a communications interface 348, and a bus 349 by which the other components of the controller 240 are in communication with each other. The processing unit 342 may be any suitable processing unit, such as a central processing unit, that executes instructions. The memory 344 is a short-term, volatile memory, such as random access memory (RAM). The storage 346 is a long-term, non-volatile storage device, such as a solid-state storage medium. The storage 346 may, for example, be a computer readable medium that includes the instructions that are executed by the processing unit 342 for implementing the devices, systems, and methods described herein. The communications interface 348 is configured to send and/or receive signals, such as for operating various other electronic components and/or receiving information therefrom.

The controller 240 may be provided in any suitable form, including, but not limited to, a microcontroller, application specific integrated circuit (ASIC), field programmable gate array (FGPA), or as separate components. The controller 240 may also be provided as an integrated unit with the motion sensing unit 230, for example, being configured as a system-on-a-chip (SOC) therewith.

The data storage 250 is configured to store the motion data that is output from the motion sensing unit 230 and the motion sensors thereof. The data storage 250 is a non-volatile, long-term storage device, such as solid-state storage. The motion data (e.g., accelerometer readings) is stored by the data storage 250 in association with a time indicator provided, for example, by a timer of the controller 240 (e.g., clock, time stamp counter). The time indicator may include known dates and times or may be another numerical indicator from which the dates and times may be associated with the collected motion data. In some embodiments, the motion data may undergo minor processing, for example, to reduce the amount of motion data stored (e.g., via data compression, filtering, and/or averaging).

The motion data may be storage in one or more secured manners and/or for privacy. For example, the motion data may be stored in an encrypted format and/or otherwise require a security key for access thereto (e.g., when transferred from the data storage 250). Still further, the motion data may be stored in an anonymous manner, for example, with the data storage not storing an identifier of the patient (e.g., patient identification number or name) and/or the wearable electronic device 100 may be configured to not receive any patient identifying information from the patient and/or the prescriber (e.g., being configured to not transfer data with devices associated with the patient and/or the prescriber). Rather, the wearable electronic device may include a device identifier (e.g., a serial number) that the prescriber and/or the distributor stores in association with a patient identifier, such the only the prescriber and/or the distributor may be able to associate the motion data, or assessment thereof, with the patient.

The data storage 250 may be any suitable size to record the time indicator and the motion data, which as described in further detail below, may account for different stages of a useful life of the wearable electronic device 100 and modes of operation. For example, the data storage 250 may be between 1 MB and 4 GB, such as between 400 MB and 4 GB, more, or less. In one illustrative, non-limiting example, if the motion data is stored in 16-bit format from three accelerometers (e.g., from a three-axis accelerometer) at 30 Hz over a minimum wear period of four weeks, the motion data will require approximately 400 MB of the data storage 250. In another illustrative, non-limiting example, if the motion data is stored in 8-bit format from three accelerometers at 1 Hz over a period of 14 days, the motion data will require approximately 4 MB of the data storage 250.

The data storage 250 may be the storage 346 of the controller 240, a separate component, or be provided as an integrated unit with the motion sensing unit 230, for example, being configured as a system-on-a-chip (SOC) therewith.

The power source 260 is configured to provide electrical power for operating the electronics 120 of the wearable electronic device 100. In one example, the power source 260 is a primary battery (i.e., a non-rechargeable battery), such as a metal-air battery (e.g., zinc-air, lithium-air, aluminum-air, or magnesium-air) or other type of primary battery (e.g., lithium, alkaline, or zinc-carbon). A primary battery may be advantageous over a secondary battery (i.e., a rechargeable battery) by being lower cost and/or by having a higher power density and, thereby, a smaller size for equivalent power capacity. Alternatively, the power source 260 may instead be or include a secondary battery (i.e., a rechargeable battery), a battery having an exchangeable electrolyte (i.e., being refillable), a capacitor, a super capacitor, and/or an energy harvester.

The power source 260 may be provided in any suitable format. In one example, the power source 260 is a coin cell battery, which may be advantageous by having a relatively small form factor (e.g., low height), being readily available, and relatively low cost.

The power source 260 is of suitable capacity to operate the wearable electronic device 100 over the useful life thereof and different modes of operation.

In the case of the power source 260 being a metal-air battery, the body 110 and/or the housing portion 112 and the power source 260 are cooperatively configured for sufficient air to reach the metal-air battery to support operation thereof (e.g., with the membrane 112b) or the material thereof otherwise being sufficiently air permeable.

Furthermore, the wearable electronic device 100 may be provided in a manner such that the metal-air battery is not activated until an event associated with the patient wearing the wearable electronic device 100. For example, the wearable electronic device 100 may be provided in packaging 402 that prevents air from reaching the metal-air until the packaging is opened (as illustrated in FIG. 4A), or provided with a removable air-impermeable barrier 404 that prevents air from reaching the metal-air battery until removed (as illustrated in FIG. 4B). In each case, the packaging 402 or the removable air-impermeable barrier 404 may include printed instructions to not open the packaging or removing the barrier until the wearable device is to be worn and/or to not wear the device if the packaging was previously opened or the barrier previously removed. The packaging 402 may further contain an oxygen-absorptive material to preserve the power source 260 and/or a moisture-absorptive material. Alternatively, as discussed below, the wearable electronic device 100 may include a light sensor 292, while the packaging 402 is opaque. Upon removing the wearable electronic device 100, the light sensor 292 receives light from the environment (e.g., environmental light) and activates the wearable electronic device to detect the patient and/or motion thereof.

In other configurations, the power source 260 may operate before the wearable electronic device 100 is worn to detect the patient (e.g., with the proximity sensor 270, as discussed below), or may be activated upon occurrence of physical trigger that may be associated with the patient wearing the device (e.g., subsequent to which the proximity sensor 270 and/or the motion detector 130 are operated). For example, the power source 260 may include a permanent switch that is closed when the device is worn, such as with an electrically insulative member being removed from between electrical contacts of the power source 260 and the other electronics 120 to close a circuit therebetween, such as upon stretching the body 110 or flexing the body 110 around the wrist of the patient. Such physical (e.g., mechanical) triggers may include breaking of a wire, connecting or disconnecting the coupling portions 114 of the body 110, a magnetic switch, and/or removal of a clip, pin, or sticker. Instead of a physical trigger, an optical trigger (e.g., removing the wearable electronic device 100 from opaque packaging) may be used to activate the wearable electronic device 100, as was described previously.

The wearable electronic device 100 and/or the electronics 120 may be configured for the power source 260 to be replaceable, for example, having spring contacts that conductively and mechanically releasably engage the power source 260. Alternatively, the power source 260 may be configured to be reusable by replacing electrolyte thereof, or by being recharged conventionally by electrically coupling a power source thereto (e.g., via wired connecting with conductive contacts, or wirelessly with a telemetry coil). As described previously, the body 110 (e.g., the housing portion 112) may include the opening 112d that is sealed by the removable segment 112c, but which provides physical access to the power source 260 for replacement or recharging thereof, or the electronics 120 may be entirely removed from the body 110 to provide physical access to the power source 260. In some embodiments, the wearable electronic device 100 and/or the electronics 120 are configured for the power source 260 to not be recharged (e.g., including no contacts or coils by which power may be transferred to the power source 260).

The wearable electronic device 100 may, in some embodiments, include the proximity sensor 270. The proximity sensor 270 may be used to determine whether to operate the motion sensing unit 230 to collect the motion data (e.g., by determining whether the wearable electronic device 100 is being worn, or a proxy indicative thereof, such as capacitance exceeding a threshold). Capacitance data may be recorded, for example, for the prescriber to assess compliance with the prescriber's instructions to the patient. The proximity sensor 270 may be operated in a patient detection mode and may be further operated in the motion data collection mode, as discussed in further detail below.

The proximity sensor 270 may be any suitable type of sensor for detecting proximity of the patient thereto. In one example, the proximity sensor 270 is a capacitive sensor. The proximity sensor 270 may be arranged on an underside of the substrate 290, such that the substrate 290 is not positioned between the patient and the proximity sensor 270 when the wearable electronic device 100 is worn by the patient. In a preferred example, a physical barrier is arranged between the proximity sensor 270 and the patient being detected thereby, which may include a portion of the body 110 (e.g., the housing portion 112).

The proximity sensor 270 is operated in a manner configured to consume relatively little power when the wearable electronic device is not worn by the patient. For example, the proximity sensor 270 may be operated for short durations (e.g., between one tenth and ten seconds) spaced apart at large intervals (e.g., between 5 minutes and four hours, such as between 10 and 20 minutes), which may be referred to as patient detection durations and patient detection intervals. The patient detection intervals may be configured relative to possible wear periods, such that the motion data will be collected for a substantial majority of the time that the wearable electronic device 100 is worn by the patient (e.g., possibly not collecting the motion data for at most a period of time equal to the patient detection interval). For example, a prescriber may prescribe the wearable electronic device 100 to be worn for one week, while the patient detection interval is for hours. While collecting the motion data, the proximity sensor 270 may continue to be operated at the patient detection interval to confirm that the wearable electronic device 100 is still being worn and is continued to be operated to collect the motion data. If the patient is no longer detected with the proximity sensor 270, the wearable electronic device 100 stops collecting the motion data until the patient is again detected therewith (e.g., operating in the patient detection mode but not the motion data collection mode).

The communications interface 280 of the wearable electronic device 100 allows data to be transferred between the wearable electronic device 100 and another computing device (e.g., of the processing facility, as discussed in further detail below). For example, the communications interface 280 enables the motion data to be transferred from the wearable electronic device 100 after being worn by the patient. The communications interface 280 may further enable receipt of data by the wearable electronic device 100, such as signals to initiate transfer of the motion data (e.g., which may include an encryption key) and/or to provide data to the wearable electronic device 100 (e.g., updated software programming by which the controller 240 operates the wearable electronic device 100).

The communications interface 280 may take any suitable form for transfer of data with the wearable electronic device 100. In a preferred example, the communications interface 280 provides wired data transfer by including conductive contacts that are configured to conductively couple to corresponding conductive contacts of mating communications interface of the other computing device, for example, to transfer data directly from the data storage 250 (e.g., a bus connected to the data storage 250). In another example, the communications interface 280 is configured for wireless communication and includes appropriate hardware (e.g., coil, antenna, or semiconductor) for sending and/or receiving data according to any suitable protocol (e.g., Bluetooth), and such hardware may also function to harvest energy (e.g., RF energy) to replenish the power source 260 or another power source. In some embodiments, the wearable electronic device 100, the electronics 120, and the communications interface 280 thereof are configured to not transfer the motion data wirelessly (e.g., including no antennas or other devices by which the data may be transferred wirelessly).

The communications interface 280 may be encapsulated by the body 110, whether being configured for wired or wireless data transfer. For example, the body 110 may include the removable segment 112c that, when removed, opens the opening 112d to provide physical access to the communications interface 280. In another example, the body 110 is configured for the electronics 120 to be removed therefrom (e.g., being cut or torn). In each such case, physical access to the communications interface 280 may require permanent deformation of the body 110 and/or prevents physical access to the communications interface 280 by the patient and prescriber.

The electronics 120 may be provide as a singular unit, for example, by being coupled to the substrate 290, such as a circuit board by which power and/or signals may transfer between the different electronic components. The substrate 290 and the electronics 120 coupled thereto may be referred to cooperatively as an electronics module.

The substrate 290 and/or the electronics module may be configured to conform to the patient in a radial direction relative to the patient (e.g., radially inward and outward relative to the wrist of the patient) and/or a tangential direction relative to the patient (e.g., along the surface of the wrist of the patient). To conform in the radial direction, in a preferred example, the substrate 290 is a flexible circuit board, which may also be referred to as flex. In such case, the substrate 290 conforms in the radial direction by bending around the wrist of the patient. Instead of or in addition to the substrate 290 being a flexible circuit board, a compressible material (not shown; e.g., a foam pad that is more compressible than the substrate 290 and/or the housing portion 112 formed therearound) is arranged between the patient and the substrate and may further be covered by material forming the body 110. In another implementation, the body 110 (e.g., the electronics housing 112a thereof) may function as the substrate 290, for example, with the various electronic components embedded therein and supported thereby with conductive pathways formed therebetween (e.g., silver, graphene, graphite, copper, or graphene-impregnated silicone), for example, via an additive or other suitable manufacturing process.

In some embodiments, the electronics module may be physically separated from the material forming the housing portion 112 of the body 110, such as with a sheet material arranged therebetween, which allows shearing motion between the electronics module and the body 110 as the band is stretched. Instead or additionally, various components of the electronics 120 may be encapsulated (e.g., in an epoxy), so as to fill voids between such electronic components that might otherwise be filled by the material of the body 110 molded there around and/or to form a smooth surface that provides for easier removal of the electronics module from the body 110.

Referring to FIG. 2B, the electronics 120 may also include a light sensor 292 in addition to the motion sensing units 230, the controller 240, the data storage 250, the power source 260, and the communications interface 280 described previously. The light sensor 292 may, for example, be a photodiode or phototransistor that changes an output thereof (e.g., current and/or voltage) according to environmental light received thereby. The light sensor 292 may, as discussed in further detail below, be used as a trigger or to otherwise determine when to change between modes and/or operation of different sensors, such as to begin operating the proximity sensor 270 (e.g., for detecting a patient), to begin operating the motion sensing unit 230 ((e.g., with the proximity sensor 270), to begin recording time indicators, to begin recording motion data, to stop and/or restart operating the proximity sensor 270, to stop and/or restart operating the motion sensing unit 230, and/or to stop and/or restart recording the motion data. As used herein, the term “begin” when used in conjunction operating various sensors and/or different modes generally refers to the first time after manufacturing and removal of the wearable electronic device 100 from the packaging 402 (e.g., after a patient receives the wearable electronic device 100) that such an operation is performed. The term “restart” generally refers to subsequent occurrences after lapse of such operations.

In one example, the controller 240 is operated in a low-power state, and the light sensor 292 outputs a signal to the controller 240 (e.g., the processor 342 thereof), which functions as a trigger or an interrupt to which the controller 240 (e.g., the processor 342 thereof) is initiated and begins to operate as a timer or clock (i.e., providing time indicators) and may further begin to operate the proximity sensor 270 to detect a patient wearing the wearable electronic device 100 (e.g., if capacitance exceeds a threshold) and/or may further begin to operate the motion sensing unit 230 to detect motion of the patient and/or to detect the patient (e.g., based on the motion detected). For example, the controller 240 may cause the power source 260 to provide power to the proximity sensor 270 and/or motion sensing unit 230 for operation thereof. Thus, after sensing environmental light with the light sensor or in response to the sensing environmental light with the light sensor), the timer begins providing the time indicators and one or more of the motion sensing unit begins sensing the motion, the proximity sensing begins operating to sense the patient, and the motion data (or other data derived therefrom) is stored in association with the time indicators. The motion data may be further processed to provide the other data (e.g., processed motion data), for example, being processed as the root mean square of three axes of motion data (e.g., acceleration in three perpendicular axes).

The timer may continue to provide the time indicators until subsequent data transfer between the data storage and a computing device. For example, when the timer begins providing the time indicators after the light sensor senses the environmental light, such time indicators will not be associated with a particular data and time of day. Thus, by continuing to operate the timer until subsequent data transfer allows for association of the time indicators with known date and time, either by transferring a known date and time to be stored in association with a time indicator on the wearable electronic device 100 or transferring the motion data or other data derived therefrom to the computing device to be stored in association with a known date and time.

The light sensor 292 and/or the motion sensing unit 230 may also be used to determine when to stop and/or restart various operations. For example, after environmental light is not sensed (e.g., above a threshold) and motion is not detected (e.g., above a threshold) and/or in response thereto, such as when the wearable electronic device 100 is placed in return mail packaging, the controller 240 may stop the motion data from being recorded and/or stop or otherwise slow operation of the motion sensing unit (e.g., by operating less often and/or at a lower frequency) to conserve power.

The light sensor 292 may also function as a signal receiver (e.g., instead of or in addition to the communications interface 280), for example, receiving instructions and/or programming from an external source (e.g., during manufacturing and/or when processing the wearable electronic device 100 to extract the motion data therefrom).

The electronics 120 may also include a light source 294, which may be configured to provide a visual indication to the patient that the wearable electronic device 100 is operating properly. The light source 294 may, for example, be a light-emitting diode that is operated intermittently, for example, being turned on one or more times (e.g., blinked or flashed) at an indicator frequency, such as once per minute. Instead or additionally, the light source 294 may operate (e.g., emit light) in response to shaking of the wearable electronic device 100 (e.g., measured by the motion sensor units 230 or the motion sensors 232). The light source 294 may be configured, in conjunction with the housing portion 112, so as to not distract the patient, such as by emitting low levels of light. For example, the light source 294 may output light at 10, 8, 5, 2, or fewer lumens.

The light source 294 may also function as a signal transmitter (e.g., instead of or in addition to the communications interface 280), for example, sending the motion data during the processing. In such case, the light source 294 may instead or additional be referred to as a signal transmitter. When transmitting signals, such as motion data signals, the light source 294 may consume relatively high power compared to when functioning as an indicator and receive power from an external power source. The light sensor 292 and the light source 294 may cooperatively function as and in place of the communications interface 280.

It should be noted that the housing portion 112 or subportion thereof may also be configured to permit light to pass therethrough (e.g., being translucent or transparent), such that environmental light may pass to the light sensor 292, for example, to activate the wearable electronic device 100 and/or to allow light from the light source 294 to pass therethrough to the environment. As mentioned, the wearable electronic device 100 may be provided to a patient in an opaque package (e.g., a system having an opaque package and the wearable electronic device in the opaque package), such that removal of the wearable electronic device 100 causes light to reach light sensors 292 for activating the wearable electronic device 100 (e.g., changing modes of operation thereof to begin detecting the patient and/or motion). The opaque packaging (e.g., the packaging 402) may, for example, be made of Mylar or other opaque polymer, which may be metallized and further function as a faraday cage that prevents the proximity sensor 270 (e.g., a capacitive sensor) from detecting objects outside the package. The package may contain the wearable electronic device 100. Alternatively, an opaque cover may be coupled to and removable from the housing portion 112 that prevents environmental light from reaching the light sensor 292.

Referring to FIGS. 2C and 2D, the electronics 120 may be provided on the substrate 290, which may be configured as a flexible circuit board (e.g., flex circuit) that is folded, rolled, and/or otherwise shaped to be contained within the housing portion 112 (e.g., a tube or canister, as illustrated in FIGS. 1E and 2D). The substrate 290 may generally include a central portion 290a and two outer portions 290b (e.g., wings) that extend outward from the central portion 290a. The central portion 290a includes connected thereto any suitable combination of the motion sensors 232, the controller 240, the data storage 250, the power source 260, the communications interface 280, the light sensor 292, and/or the light source 294. The two outer portions 290b form portions of the proximity sensor 270, for example, forming electrodes of a capacitive sensor.

The substrate 290 is collapsed and contained inside the housing portion 112, for example, with the ends of the housing portion 112 being sealed. For example, the substrate 290 may be inserted into the housing portion with the outer portions 290b being positioned adjacent interior sides of the housing portion 112, for example, such that the outer portions 290b extend around a majority of an inner peripheral surface of the housing portion 112, such as at least 50%, 60%, 75%, 80% or more. That is, the combined widths of the outer portions 290b, or the width of singular outer portion 290b in the case of one outer portion 290b forming the electrode, is greater than 50%, 60%, 75%, 80% or more of the periphery of an inner surface of the housing portion 112. The central portion 290a may be arranged between the two outer portions 290b. For example, the substrate 290 may be folded in the shape of the letter “W” or “Z” (e.g., having a W-fold or a Z-fold, respectively).

The substrate 290 may further include conductive contacts (e.g., positive and negative contacts) proximate the ends of the housing portion 112 that are configured to receive power from an external source. For example, during processing of the wearable electronic device 100, the seals in the ends of the housing portion 112 may be pierced and, thereby, mechanically deformed with corresponding conductive contacts that provide power to the electronics 120, for example, to provide power to the light source 294 and/or the communications interface 280 to transmit signals with the motion data stored by the wearable electronic device 100.

The packaging 402 may, instead of or in addition to preventing air from reaching the power source 260 (i.e., in the case of being a metal-air battery), may be configured to provide further functions, such as being configured as a faraday cage, be re-closeable, include return shipping label, and/or include markings 406 to facilitate processing. When configured as a faraday cage, the packaging 402 may be formed of a metallized material that prevents electromagnetic interference with the wearable electronics device 100 (e.g., the electronics 120 thereof, such as the proximity sensor 270). The packaging 402 may be further configured for the patient to send the wearable electronic device 100 to the processing facility, for example, being re-closeable (e.g., with an included adhesive layer) and/or including a shipping label (e.g., a pre-paid shipping label). In the case of the power source 260 being a metal-air battery, the packaging 402 may be configured to permit air to reach the power source 260 when packaging 402 is re-closed. The packaging 402 may also include markings 406, which may be provide on the shipping label, or otherwise to facilitate processing of the wearable electronic device 100. The markings 406 may be machine-readable to orient the packaging 402 for physical processing thereof (e.g., to open and remove the wearable electronic device 100) and/or to identify the wearable electronic device 100 (e.g., including a unique identifier associated with the wearable electronic device 100, such as a serial number, shipping tracking number, and/or bar or other QR code associated therewith).

As referenced above, the wearable electronic device 100 may configured to avoid influencing behavior of the patient, present few and/or low barriers to use, and be relatively low cost. To achieve these ends, the wearable electronic device 100 may not include various types of electronic components, limit operation of such components, or prevent or otherwise limit interaction with such components if included. Such excluded or limited-use electronic components could be output components, input components, and/or electronic interface components.

The wearable electronic device 100 may not include, or may limit operation of, output devices that would otherwise provide outputs that could be sensed by the patient directly from the wearable electronic device 100, which might draw the attention of the patient to the wearable electronic device 100 and, thereby, influence behavior of the patient.

Output devices may be generally categorized as visual output devices, audio output devices, or tactile output devices, each of which are output devices that are selectively operated to provide an output. For example, the wearable electronic device 100 may not include a display screen, may not include a light, may include neither a display screen nor a light, or may not include any visual output device, which would otherwise be selectively operable to provide an output that could be sensed visually by the patient. Instead of or in addition to not including visual output devices, the wearable electronic device 100 may not include a speaker, may not include a buzzer, may include neither a speaker nor a buzzer, or may not include any audio output device, which would otherwise be selectively operable to provide an output that could be sensed aurally by the patient. Instead of or in addition to not including the visual output devices and/or audio output devices, the wearable electronic device 100 may not include any tactile output device that would otherwise be selectively operable to provide an output that could be sensed tactilely by the patient.

In one embodiment, the wearable electronic device 100 may include a simplified visual output device (e.g., the light source 294) and no other visual output devices. The simplified visual output device includes three or fewer lights (e.g., one LED) that blinks to provide one or more binary indicators (e.g., whether motion data is being collected or not, whether the power source 250 has reached a low power threshold or not, and/or whether the data storage 260 has reached a data storage threshold or not).

In other embodiments, the wearable electronic device 100 may include one or more of the visual output devices, the audio output device, or the tactile output devices, while being configured to output limited information or being configured to be selectively operated in limited circumstances. For example, the wearable electronic device 100 may be configured to not output any indication of the activity of the patient (e.g., indicating that motion data has started to be collected, is being collected, and/or has stopped being collected, or a status of the power source 260). In another example, the wearable electronic device 100 may be configured to provide limited types of outputs, such as in relation to only one or more of the following: power or operation of the wearable electronic device 100 (e.g., confirming the device is powered on), recording motion data (e.g., upon starting, during, and/or upon completion thereof), the power source (e.g., an indicator of remaining power), transfer of the motion data (e.g., upon initiation, during, and/or completion thereof), or the time (e.g., the time of day and/or data).

By not including any direct output devices, not including certain types of direct output devices, not providing an activity-related output, or providing outputs in relation to only limited types of outputs, the wearable electronic device may limit the circumstances in which the attention of the patient is drawn to the wearable electronic device, which might otherwise influence behavior of the patient. Further, by not including various or any direct output devices, the weight, size, and cost may be lessened as compared if the wearable electronic device 100 were to include such direct output devices.

The wearable electronic device 100 may not include, or may limit operation of, various direct input devices that could otherwise allow or require the patient to provide intentional user inputs to the wearable electronic device. Requiring or allowing such inputs from the patient might otherwise draw the attention or invite interaction of the patient, thereby influencing behavior of the patient. Further, requiring inputs from the patient or the prescriber may increase the real or perceived barriers to use of the wearable electronic device 100.

Input devices may be generally categorized as optical input devices, audio input devices, or physical input devices, each of which are devices that are configured to receive intentional inputs directly from humans. Intentional inputs are to be distinguished from passive behaviors that are intended to be observed (e.g., motion of the patient). For example, the wearable electronic device 100 may not include any optical input device, which might otherwise be configured to optically receive inputs (e.g., gestures) from the patient or the prescriber. Instead of or in addition to not including the optical input device, the wearable electronic device 100 may not include an audio input device (e.g., a microphone), which might otherwise be configured to audibly receive inputs (e.g., voice commands) from the patient or the prescriber. Instead of or in addition to not including the optical input device or the aural input device, the wearable electronic device 100 may not include any physical input device, which might otherwise be configured to receive physically detect intentional physical inputs (e.g., button presses, or swipes or other gestures). It should be noted that the proximity sensor 270, in the use described herein of detecting capacitance indicative of the wearable electronic device 100 being worn by the patient, is not considered herein to be a physical input device, because sensed capacitance is incidental to the passive behavior of the patient wearing the wearable electronic device 100 and not an intentional user input.

In other embodiments, the wearable electronic device 100 may include one or more of the optical input devices, the audio input devices, or the physical input devices, which are configured to not receive intentional inputs from the patient.

By not including any direct input devices, or by receiving direct input in relation to only limited circumstances that require or invite interaction of the patient, the wearable electronic device 100 may limit the circumstances in which attention is drawn or required of the patient or the prescriber, which might otherwise influence behavior of the patient or present a barrier to use for the patient or the prescriber. Further, by not including various or any direct input devices, the weight, size, and cost may be lessened as compared to if the wearable electronic device 100 were to include such output devices.

The wearable electronic device 100 may not require electronic interaction (e.g., transferring power and/or data, or any other electronic input or output) by the patient or the prescriber with the wearable electronic device 100, such as with the power source 260 (e.g., to charge or replace a battery) and/or the communications interface 280 (e.g., to operate the wearable electronic device 100, to retrieve, process, or view the motion data, or provide other intentional inputs to the wearable electronic device 100). Furthermore, the wearable electronic device 100 may be configured to not allow the patient or the prescriber to interact electronically with the wearable electronic device 100 with another electronic device.

The power source 260, as will described in further detail below, is configured to provide the wearable electronic device 100 with sufficient capacity to supply power over the useful life of the wearable electronic device 100. As a result, none of the patient, the prescriber, or other custodian (e.g., a distributor of the wearable electronic device 100) is required to maintain or replace the power source 260. Furthermore, as described previously, physical access to the power source 260 may be hindered by requiring the body 110 to be damaged or the removal of a sacrificial component to gain physical access thereto. As a result, maintaining the power source 260 does not provide any barrier to use by the patient, the prescriber, or other custodian.

Regarding the communications interface 280, the wearable electronic device may be configured to prevent, not require, or provide for limited electronic interaction with the communications interface 280. In one example, the wearable electronic device prevents or substantially hinders data transfer with the communications interface 280 physically (e.g., preventing physical access as just described for the power source 260 or by using a proprietary data connector) or electronically (e.g., by requiring an access code or encryption key to transfer and/or read the motion data). Further, no interaction with the communication interface 290 may be required to initiate collection of motion data (e.g., instead collecting motion data upon power delivery with a metal air-battery, detection with the proximity sensor 270, or other trigger inherent to use of the wearable electronic device 100) or otherwise set up the wearable electronic device 100 for use (e.g., no data is provided by the patient, the prescriber, or the custodian to the wearable electronic device 100, such as identification information of the patient or prescriber).

In another example, the wearable electronic device 100 is configured to send and/or receive signals and data from another electronic device (e.g., wirelessly via the communications interface 280). For example, the wearable electronic device 100 may be configured output the motion data to an electronic device associated with the patient and/or may be configured to receive inputs from the user via the other electronic device (e.g., subjective inputs of pain experienced by the patient), such as a smartphone or a docking station (e.g., which may further charge the power source 260).

As referenced above, the wearable electronic device 100 and, in particular, the electronics 120 and operations thereof may be configured according to a useful life, which may be considered to generally include a wear period, a pre-wear period, and a post-wear period. The wear period is that during which the wearable electronic device 100 is or should be (e.g., is prescribed to be) worn by the patient. The pre-wear period is that prior to the wearable electronic device 100 first being worn by the patient. The post-wear period is that after the patient is done wearing the wearable electronic device 100. Various aspects of the wearable electronic device 100 may be configured according to the pre-wear, the wear, and the post-wear periods (e.g., to ensure that the wearable electronic device is operable over such periods). That is the pre-wear, wear, and post-wear periods are design considerations. The wearable electronic device 100 may operate in various different modes that generally correspond to the nature of the different wear periods, but the wearable electronic device may not necessarily expressly determine whether the wearable electronic device is worn in such periods.

The wear period is a period of time during which the patient wears the wearable electronic device 100 and the electronics are operated to collect the motion data. Collecting the motion data is considered to include sensing motion with the motion sensing unit 230 and storing the motion data with the data storage 250. The wear period may be prescribed by the prescriber or otherwise determined (e.g., by actual wear of the patient). The wear period may, for example, be three days, one week, one month, or three months. For example, a prescriber may provide a patient with four of the wearable electronic devices 100 prescribed to be worn in successive one-week wear periods.

The prescriber may prescribe a wear period that is within a minimum wear period of the wearable electronic device 100. The wearable electronic device 100 may be configured according to the minimum wear period, which is a predetermined minimum amount of time over which the wearable electronic device 100 is configured to collect the motion data. The minimum wear period may, for example, be between one week and four months, such as approximately one week, two weeks, four weeks, or approximately eight weeks, or other suitable time period that accounts for expected variances in wear periods desired by prescribers.

The minimum wear period is a design value that may be exceeded in use, but which is limited by capacity of the power source 260 or capacity of the data storage 250 in conjunction with operation of the other electronics 120 and other factors. Those other factors include operation of the various sensors, the rate at which the motion data is collected (e.g., frequency, number of sensors, and storage format), programmatic operations (e.g., stopping collecting motion data after a predetermined period or upon a low battery condition), and environmental characteristics (e.g., temperature), among others. The capacities of the power source 260 and the data storage 250 are discussed in further detail below with respect to first and second implementations of the electronics 120 of the wearable electronic device 100.

The pre-wear period is a period of time before the patient begins wearing the wearable electronic device 100 and generally after manufacture of the wearable electronic device 100. During the pre-wear period, the wearable electronic device 100 may fill the data storage 250 and consume power at relatively low rates as compared to during the wear period. For example, the wearable electronic device 100 may be configured to not operate the motion sensing unit 230 and/or not collect the motion data during the pre-wear period, such as by not activating the power source 260 (e.g., to not operate the proximity sensor 270 and/or the motion sensors 232) or by first requiring detection of the patient with the proximity sensor 270.

The wearable electronic device 100 may be configured according to a minimum pre-wear period, which is a predetermined minimum amount of time (e.g., a shelf life) after which the wearable electronic device 100 is configured to collect the motion data for at least the minimum wear period. The minimum pre-wear period may, for example, be between one and three years, more or less. The wearable electronic device 100 may be provided with an indicator of the minimum pre-wear period, for example, being provided in packaging that includes a printed expiration or “use by” date.

The post-wear period is a period of time after the patient wears the wearable electronic device 100 and generally before the motion data is transferred from the wearable electronic device 100 and/or until remanufacturing of the wearable electronic device 100. During the post-wear period or portions thereof, motion data collection and power consumption may, in some implementations, be reduced as compared to the wear period depending on the configuration of the wearable electronic device 100.

The wearable electronic device 100 may be configured according to a minimum post-wear period, which is a predetermined minimum amount of time during which various operations may be maintained. The minimum post-wear period generally accounts for the time between the user wearing the device and subsequent processing of the device, which may include transit time to the processing facility (e.g., for shipping), delay between the user wearing and shipping the device, and any subsequent processing delays or times. For example, the minimum post-wear period may be between one week and two months, such as one month, more or less.

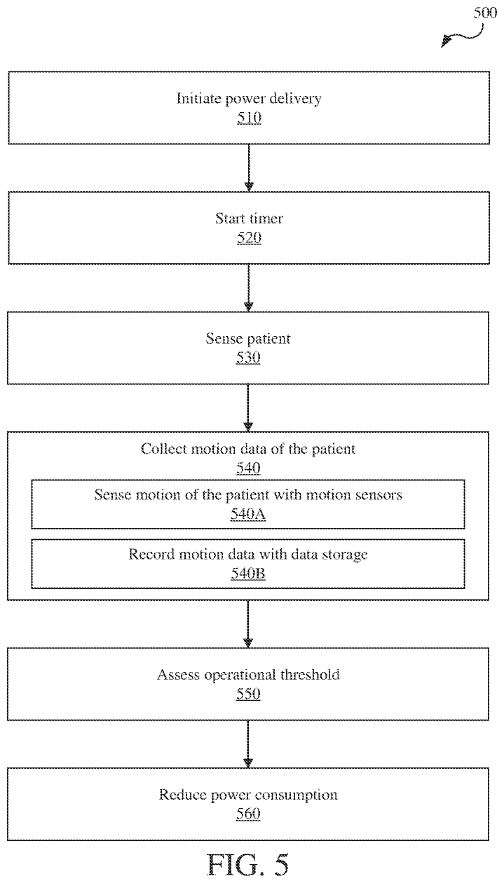

With further reference to FIG. 5, the wearable electronic device 100 may be configured to implement a method 500 for motion data collection of a patient. The wearable electronic device 100 is configured to collect motion data upon one, the other, or both of operation of the power source 260 or detection of the patient with the proximity sensor 270.

Before the wearable electronic device 100 is worn by any patient, the wearable electronic device 100 is operated in one, the other, or both of a power-off mode or a patient detection mode, which generally correspond to the pre-wear period. In the power-off mode, which may also be referred to as a power-off state, the power source 260 is not operated, such that the various other components of the electronics 120 are not operated. A timer is not operated in the power-off mode. The power source 260 may be initially operated in various manners described previously, which may include exposing a metal-air battery to air (e.g., removing the wearable electronic device 100 from air-impermeable packaging or upon removing an air-impermeable cover from the wearable electronic device 100), upon detecting light with the light sensor 292, or upon a physical event associated with the patient first wearing the wearable electronic device 100 (e.g., closing a permanent or repeatable switch incidental to motion manipulation of the wearable electronic device 100).

In the patient detection mode, a timer (e.g., a time step counter) is operated, and the proximity sensor 270 is operated at the patient detection interval to assess whether the wearable electronic device 100 is being worn by a patient. In the patient detection mode, the motion sensing unit 230 may not be operated and the motion data not be collected, or alternatively, the motion sensing unit 230 may be operated at spaced apart intervals (e.g., the patient detection interval) and/or the motion data recorded to serve as an alternative or secondary indicator of the patient wearing the wearable electronic device 100. Proximity data may, in some implementations, be stored in association with the time indicator. If the patient is not detected, the proximity sensor 270 is continued to be operated at the patient detection interval to assess whether the wearable electronic device 100 is being worn by a patient. If the patient is detected (e.g., as determined with the proximity sensor 270), the wearable electronic device 100 begins operating in a motion data collection mode. It should be noted that the wearable electronic device 100 may first be worn by a patient for a period of time, possibly as long as the patient detection interval, before the proximity sensor 270 is operated and the wearable electronic device 100 is changed to operate in the motion data collection mode. The wearable electronic device 100 may be packaged and/or otherwise configured to prevent inadvertent detections of patients, such as with the packaging physically preventing such false positives and/or having higher sensing thresholds that provide greater certainty of patient detection.

As referenced above, the wearable electronic device 100 may be configured to initially operate in one, the other, or both of the power-off mode or the patient detection mode. If configured to operate initially the power-off mode but not the patient detection mode, for example if the wearable electronic device 100 does not include the proximity sensor 270, the wearable electronic device 100 begins operating in the motion data collection mode upon operating the power source 260. In such case, the timer is not associated with a known date and time. Operating initially in the power-off mode (e.g., being provided in a power-off state) allows for an extended pre-wear period (e.g., shelf-life) before the wearable electronic device 100 is used but may require continuing to operate the timer for the duration of the post-wear period for the motion data to be later associated with known dates and times.

If configured to operate in the patient detection mode but not initially in the power-off mode, the wearable electronic device 100 is initially operated in the patient detection mode (e.g., generally upon manufacturing of the wearable electronic device 100) and, subsequently, in the motion data collection mode upon detecting the patient. In such case, the timer is associated with known date and time, which allows the motion data to be initially stored in or for association with known dates and times and further allows for the wearable electronic device 100 to be in the power-off mode for the post-wear period.

If configured to operate initially in both the power-off mode and the patient detection mode, the wearable electronic device 100 begins operating in the power off-mode, subsequently in the patient detection mode upon operating the power source 260 and then in the motion data collection mode upon detecting the patient. In such case, the timer not associated with a known date and time, which may require continuing to operate the timer for the duration of the post-wear period for the motion data to be later associated with known dates and times.

In the motion data collection mode, the timer is operated and the motion data is collected in association with time indicators therefrom (e.g., counter values and/or known dates and times). The time at which the patient is first detected with the proximity sensor 270 generally corresponds to the beginning of the wear period. In the motion data collection mode, the motion is sensed at a suitable sensing frequency (e.g., between 0.01 and 60 Hz, such as 0.1 to 30 Hz, more or less). The motion data is output from the motion sensors 232 to be stored by the data storage 250.

If not configured to operate in the patient detection mode, the wearable electronic device 100 is operated in the motion data collection mode until the motion data is transferred therefrom. Because the time indicators do not include known date and time information, the time indicators are recorded until the motion data is transferred and a known date and time can be associated with the last recorded time indicator, thereby allowing the time indicators and the motion data that were previously recorded to be back calculated with known dates and times. The motion data is, thus, continued to be collected regardless of whether the wearable electronic device 100 is worn by the patient.

As an alternative to operating in the motion data collection mode until the motion data is transferred, the wearable electronic device 100 may be operated in the motion data collection mode until the earlier of reaching an operational threshold, such as a time threshold (e.g., a pre-determined duration for collecting the motion data, such as the minimum wear period), a data threshold is reached (e.g., an accumulated amount of collected motion data or a remaining amount of data storage capacity, which may include reaching the total capacity of the data storage), or a power threshold (e.g., remaining battery life, which may include depleting all available power from the power source 260).

After reaching the operational threshold, the wearable electronic device 100 is operated in a low-power mode in which the timer is continued to be operated, while the motion sensing unit 230 is not operated and the motion data is not collected. The operational threshold may generally correspond to the minimum wear period, while the period thereafter may generally correspond to the post-wear period. However, it should be understood that wearable electronic device 100, by not sensing whether the wearable electronic device 100 is being worn, is operated in the motion data collection mode and the low-power mode regardless of whether the wearable electronic device 100 is being worn.

If configured to operate in the patient detection mode, the wearable electronic device 100 may be configured to return to operating in the patient detection mode upon not detecting the patient with the proximity sensor 270 or not detecting motion with the motion sensors 232. In one example, while in the motion data collection mode, the proximity sensor 270 may continue to be operated at the patient detection interval, and the wearable electronic device 100 continues to operate in the motion data collection mode if the patient is again detected or will return to operating in the patient detection mode if the patient is not detected. In another example, the proximity sensor 270 is not operated in the motion data collection mode, and the wearable electronic device 100 may return to the patient detection mode according to the motion sensed with the motion sensing unit 230. For example, upon detection of little or no motion for a predetermined amount of time (e.g., the patient detection interval), the wearable electronic device 100 returns to the patient detection mode with the proximity sensor 270 again being operated at the patient detection interval.

As an alternative to returning to the patient detection mode, the wearable electronic device 100 may be configured to operate in the motion data collection mode until the motion data is transferred therefrom (e.g., as described above), or until the earlier of the motion data is transferred from therefrom or an operational threshold is reached at which point the wearable electronic device 100 is operated in the low power mode (as described above) and in which the proximity sensor 270 may also not be operated. In those configurations in which the wearable electronic device 100 in which the timer is associated with known dates and times, the operational threshold may be remaining power (e.g., turning off the wearable electronic device 100).

The power source 260 and the data storage 250 are configured with sufficient capacity to operate in those of the power-off mode, the patient detection mode, the motion data collection mode, and the low-power mode that the wearable electronic device 100 is configured to operate in. The power source 260 has a capacity that accounts for the power-off mode and any self-discharge then occurring and/or operation of the patient detection mode (e.g., sufficient power for the minimum pre-wear period), the motion data collection mode and power draw from the electronics then occurring (e.g., sufficient power for operating the motion sensing unit 230 and the controller 240, as well as the proximity sensor 270 if so configured, for the minimum wear period), and the low-power mode and power draw from the electronics 120 then occurring (e.g., no power, or sufficient power for reduced operations, such as operating the timer, for the minimum post-wear period). The data storage 270 has a capacity that accounts for the patient detection mode and data storage then occurring (e.g., sufficient storage for the time indicators for the minimum pre-wear period), and the motion data collection mode and data storage then occurring (e.g., sufficient storage for the time indicators and the motion data for the minimum wear period).

Referring still to FIG. 5, a method 500 is provided for operating a wearable electronic device, such as the wearable electronic device 100. The method 500 generally includes initiating power delivery 510, starting a timer 520, sensing a patient 530, collecting motion data 540 of the patient, assessing an operational threshold 550, and reducing power consumption 560. In some implementations of the method 500, the method 500 may omit the operation of sensing the patient 530, may repeat the operation of sensing the patient 530 after the operation of collecting of the motion data 540, may omit the operation of assessing the operational threshold 550, and/or may omit the operation of reducing the power consumption 560.

The initiating of the power delivery 510 includes first providing power from a power source, such as the power source 260, to electronics that include motion sensors, such as the motion sensing unit 230, and a controller, such as the controller 240, and may also include a proximity sensor, such as the proximity sensor 270.

The wearable electronic device 100 may be provided to the patient in a power-off mode in which case the initiating of the power delivery 510 may be performed upon providing air to a metal-air battery that forms the power source 260 (e.g., upon the patient opening air-impermeable packaging containing the wearable electronic device 100, or removing an air-impermeable cover from the wearable electronic device 100), closing a switch by which the power source 260 provides power to the other components of the electronics 120 (e.g., from an action generally associated with first wearing the wearable electronic device 100), or other physical, mechanical, and/or light operated manners described previously (e.g., based on the light sensor 292). In the case of the wearable electronic device 100 being provided in the patient detection mode, the initiating of the power delivery 510 is performed during manufacturing of the wearable electronic device 100 (e.g., upon providing or connecting the power source).

The starting of the timer 520 is performed with a controller, such as the controller 240 (e.g., a clock thereof), for example, by the initiating of the power delivery 510 thereto. If the wearable electronic device is provided to the patient in the power-off mode, the timer is not associated with a known date and time (e.g., operating as a counter). If the wearable electronic device is provided to the patient in the patient wear mode, the timer is associated with a known date and time from the manufacturing process. The timer is continued to be operated during the subsequent operations (e.g., until the motion data is transferred from the wearable electronic device, or until the power source runs out of power or is otherwise operated to not provide power to the controller).

The sensing of a patient 530 is performed with a proximity sensor, such as the proximity sensor 270 (e.g., a capacitive sensor), and the controller. If a patient is not detected (e.g., if capacitance does not exceed a threshold value), the sensing of the patient 530 is repeated at a spaced apart interval, such as the patient detection interval described previously.

The sensing of the patient 530 may also include sensing motion with a motion sensing unit, such as the motion sensing unit 230. For example, the motion sensing unit may be operated at the spaced apart interval (e.g., in conjunction with the proximity sensor), the motion data being used in conjunction with the proximity data to determine whether the patient is wearing the wearable electronic device.

If the patient is not detected, the sensing of the patient 530 is repeated at the spaced apart interval. If the patient is detected, the collection of the motion data 540 of the patient is started.

The collecting of the motion data 540 of the patient is performed with one or more motion sensors, such as the motion sensors of the motion sensing unit 230, and a data storage, such as the data storage 250, as operated by the controller. The collecting of the motion data 540 generally includes sensing motion 540A of the patient with the motion sensors and storing the motion data 540B in the data storage in association with the time indicators output by the timer. The sensing of the motion 540A and the storing of the motion data 540B may be performed continuously (e.g., as opposed to spaced apart intervals) at a suitable sensing frequency (e.g., between 0.01 and 60 Hz, such as between 0.1 and 30 Hz, such as 30 Hz, or 10 Hz, more or less), or may be performed at such a frequency during motion sensing time periods (e.g., for between 2 seconds and 30 seconds, such as for 10 seconds) at motion sensing intervals (e.g., at between 30 seconds and 10 minutes, such as every 1 minute). Between the collecting and the storing, the motion data may also be processed, for example, calculating the root mean square of acceleration measured in each of three axis, the root mean square then being stored.

In the case of the method 500 including the operation of the sensing of the patient 530, the collecting of the motion data 540 may be performed simultaneous with the sensing of the patient 530 in which case the collection of the motion data 540 may be stopped when the patient is not detected and/or when motion is not detected with the sensing of the motion of the patient 530 then being repeated.

In the case of the method 500 either including or not including the operation of the sensing of the patient 530, the method 500 may further include the operation of assessing the operational threshold 550, which is performed with the controller at any suitable frequency. For example, the controller may compare an operational parameter (e.g., elapsed time, data stored, power remaining) against the operational threshold, which may be a time threshold (e.g., time during which the motion data is collected), data storage threshold (e.g., accumulated stored data or remaining storage capacity), or a power threshold (e.g., remaining power). If the threshold is not met, the wearable electronic device continues the collecting of the motion data 540. If the threshold is not met, the wearable electronic devices reduces the power consumption 560.

The reducing of the power consumption 560 includes reducing the rate of power consumption of the electronics, such as the electronics 120, such as with the controller. The reducing of the power consumption 560 may include operating in the low power mode (as described above) by stopping the collecting of the motion data 540, thereby stopping power consumption by the motion sensors. The timer is continued to be operated after reducing the power consumption 560.

Alternatively, the collecting of the motion data 540 may be performed until either the power source is depleted of energy or the motion data is transferred from the data storage.

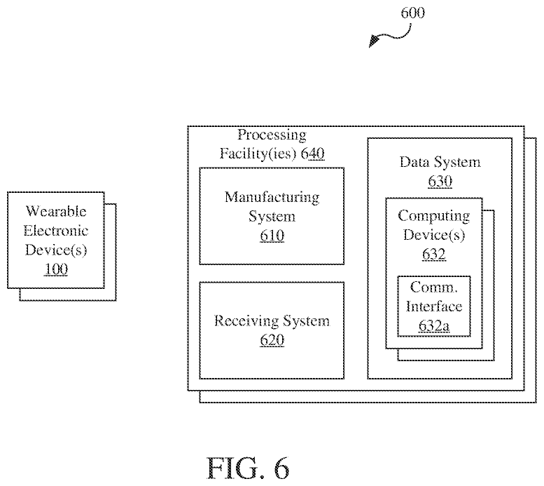

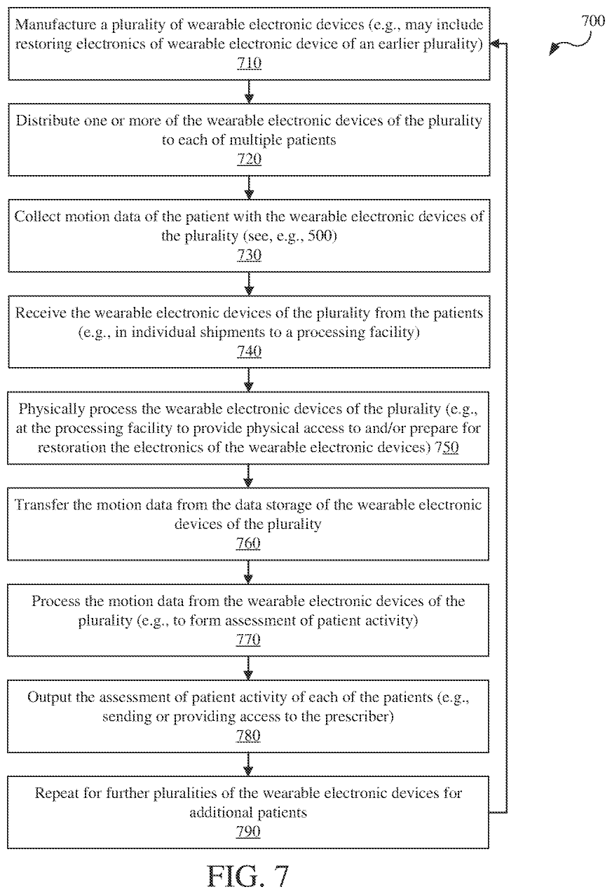

Referring to FIGS. 6 and 7, a patient activity assessment system 600 and a method 700 are provided for distributing and processing multiple of the wearable electronic devices 100 (e.g., thousands of the wearable electronic devices 100). The patient activity assessment system 600 and the method 700 are configured to distribute the wearable electronic devices 100, which includes manufacturing (or re-manufacturing) the wearable electronic devices 100 and providing the wearable electronic devices 100 to the patients. The processing of the wearable electronic devices 100 includes transferring the motion data, processing the motion data, and providing a report of the motion data to the prescriber (e.g., a physician), and may also include manufacturing or remanufacturing additional ones of the wearable electronic devices 100 from those that have been already processed (e.g., re-using the electronics 120 and possibly reusing or recycling the body 110). In some implementations of the patient activity assessment system 600 and the method 700, each of the wearable electronic devices 100 is configured as a limited-use (e.g., one-time) use device that is to be worn by only one user and over a limited period of time (e.g., up to the minimum wear period).

The patient activity assessment system 600 generally includes the wearable electronic devices 100, a manufacturing system 610, a receiving system 620, and a data system 630, which may be located at one or more processing facilities 640 to which patients send the wearable electronic devices 100 after being worn. Various functions of the manufacturing system, 610, the receiving system 620, and/or the data system 630 may be performed manually, and/or automatically. The manufacturing system 610 is configured to manufacture the wearable electronic devices 100, which may include restoring the electronics 120 of the wearable electronic device 100 that was previously worn for use by another patient. The receiving system 620 is configured to receive and mechanically process the wearable electronic devices 100 and may include, for example, automated or manually-performed processes for physically preparing the wearable electronic devices 100 for transferring the motion data therefrom or remanufacturing into another wearable electronic device 100 (e.g., removing the electronics 120 from the body 110, or by removing the removable segment 112c from the body 110 for physical access to the electronics 120). The data system 630 is configured to transfer the motion data, process the motion data, and output analyzed motion data.

The manufacturing system 610 is configured to manufacture the wearable electronic devices 100, as previously described, which may include manufacturing the wearable electronic devices 100 from new components and/or restoring the electronics 120 the wearable electronic devices 100 that were previously worn for use by another patient. In the case of manufacturing from new components, the manufacturing system 610 may be located at the same or different processing facilities 640 than the receiving system 620 and the data system 630. In the case of restoring the electronics 120, the manufacturing system may be located at the same processing facility 640 as the receiving system 620. Restoring the electronics 120 may include restoring the power source 260 (e.g., by replacing, recharging, or refilling a battery that forms the power source), restoring the data storage 250 (e.g., by removing previously-stored motion data, mapping defects therein, writing an encryption key that is shared with the data system 630, and/or encoding a new unique identifier or serial number), and resealing the electronics 120 in a body 110 (e.g., by replacing the removable segment 112c in the existing body 110, or by molding the electronics 120 into a new one of the bodies 110 (e.g., in the housing portion 112 thereof)). In the case of the power source 260 being a metal-air battery, the power source 260 is also sealed from air, for example, by sealing the wearable electronic device 100 in air-impermeable packaging or by applying a removable air-impermeable seal thereto. The manufacturing system 610 may also individually package each of the wearable electronic devices 100 in the packaging 402, which may include printing a date related to the useful life of the wearable electronic device 100 (e.g., an expiration or use-by date, or a date of manufacturing and an expiration or use-by period), printing a unique identifier associated with the particular wearable electronic device 100, and/or providing a return mail package, label, or instructions (i.e., for the patient to later send the wearable electronic device to the processing facility 640).

Restoring the electronics 120 may further include various inspections and/or testing of the electronics, such as testing of the data storage 250, testing and/or calibration of the various sensors (e.g., the motion sensors of the motion sensing unit 230 and/or the proximity sensor 270), and testing of the power source 260 (e.g., the battery).

The manufacturing system 610 may also process the body 110, for example, for reuse (e.g., disinfecting and/or cleaning) or recycling (e.g., grinding the material forming the body 110).

After manufacturing, the wearable electronic devices 100 are provided by distributors to the patients. A prescriber (e.g., a physician) may prescribe that one of the wearable electronic devices 100 be worn by a patient for a prescribed wear period (e.g., less than the minimum wear period, such as being prescribed for one week or other desired duration), or that a patient wear multiple of the wearable electronic devices in successive prescribed wear periods (e.g., four successive one week periods). The distributor provides the one or more wearable electronic devices 100, as prescribed by the prescriber, to each of the patients. The distributor may be the prescriber (e.g., the physician or practice), which may be referred to as a prescribing distributor, or by another distributor (e.g., a pharmacy), which may be referred to as a non-prescribing distributor. The distributor may associate a device identifier (e.g., a serial number) with a patient identifier (e.g., a patient identification number of name) and with a prescriber identifier (e.g., a physician or practice identification number, username, or given name). For example, the distributor may associate the wearable electronic device with the patient by recording the device identifier in a record of the patient (e.g., a medical record), which allows the prescriber to later associate the motion data from the wearable electronic device 100 with the patient that wore the wearable electronic device 100. For example, the distributor may associate the wearable electronic device with the prescriber by providing the device identifier and the prescriber identifier to the data system 630, such as through a simple message (e.g., an email) or a dedicated portal (e.g., a computer). This allows the data system 630 to provide the motion data to the prescriber, or to allow the prescriber access to the motion data or an assessment thereof (e.g., an activity assessment).

The receiving system 620 is configured to receive and prepare the wearable electronic devices 100 for data transfer and/or for restoration. The receiving system 620 may include various automated and/or manually performed operations, which may include physically preparing the wearable electronic devices 100 for the data system 630 to later physically connect thereto for data transfer. This may include removing the electronics 120 from the body 110, removing the removable segment 112c, or inserting conductive probes through the body 110 to the communications interface 280, each of which may involve permanent deformation to the body 110. In the case of the electronics 120 being removed from the body 110, material from the body 110 may be recycled and used to form a new one of the bodies 110.

For example, the receiving system 620 may include one or more optical readers for reading the unique identifier on the packaging 402 of the wearable electronic device 100 and/or reading orientation markings on the packaging 402. The receiving system 620 may also include an orienting system and an opening system, the orienting system being configured to orient the packaging 402 according to the orientation markings for the opening system to open the packaging 402 and remove the wearable electronic device. The receiving system 620 may further include one or more additional optical readers for reading orientation markings on the wearable electronic device 100 and/or one or more orientation devices for orientation the wearable electronic device 100 for the data system 630 to connect thereto and/or for a cutting system to remove the body 110 from the electronics 120.

The data system 630 includes one or more computer systems that are each configured to perform one or more of transferring the motion data from the wearable electronic devices, processing of the motion data, or outputting processed motion data, such as a patient activity assessment, to the prescriber. In a simplified example, a single one of the computing devices 632 is described herein and includes a communications interface 632a for transferring data therewith (e.g., from the data storage 250 and output to the prescriber) and which perform the data transfer, processing, and output functions, but it should be understood that multiple different computers may be utilized to perform one or more of such functions (e.g., multiple computers that each perform all three functions, or multiple computers that each perform one or two of the functions).

The computing device 632 includes the communications interface 632a that connects to the communications interfaces 280 of the wearable electronic devices 100 to transfer the motion data therefrom. The communications interface 632a of the computing device 632 may connect physically (e.g., conductively) or wirelessly to the communications interfaces 280 of the wearable electronic devices 100. As referenced above, the wearable electronic devices 100 may store the motion data in an encrypted and/or compressed format or require a security key to transfer the motion data, while the data system 630 is configured to provide or otherwise utility a security key for transferring and/or decrypting the motion data of the wearable electronic devices.

The data system 630, with the computing devices 632, processes the motion data transferred from the wearable electronic devices 100, which includes analyzing the motion data to generate patient activity assessments for each of the patients. As referenced above, the motion data may include measured values of the motion sensed with the motion sensor without further analysis thereof performed by the wearable electronic device 100. The processing of the motion data may be performed in any suitable manner to quantitatively describe activity of the patient over time, for example, using common activity metrics (e.g., steps) or uniquely determined activity metrics with suitable algorithms. If the motion data was recorded in association with time indicators that do not include the date and time, the processing may include associating the motion data with specific dates and times based on the association of one of the time indicators with a known date and time (i.e., current date and time) during the processing of the motion data. The processing of the motion data may also include identifying times at which the wearable electronic device 100 was worn by the patient and not worn by the patient (e.g., by recognizing patterns of movement associated with patient wear as contrasted with patterns of movement associated with processing or transport of the wearable electronic device).

The data system 630, with the computing devices 632, outputs the analyzed motion data to the prescriber in any suitable manner, such as via email with static reports or via an interface (e.g., a portal) that allows manipulation of the analyzed motion data. The assessed motion data may be output as a patient activity assessment, which may include a quantification of the activity of the patient against time. The motion data may be output in an anonymized manner, for example, in association with an identifier of the wearable electronic device 100 (e.g., a serial number).

As referenced above, after the motion data is transferred from the data storage 250, the electronics may be restored for reuse in the wearable electronic device 100 by another patient, or to be incorporated into a new one of the wearable electronic device 100 (e.g., with a new one of the bodies 110 being molded around the electronics 120 to form a new one of the wearable electronic devices 100).

Referring to FIG. 7, a method 700 is provided for assessing activity of multiple patients. The method 700 generally includes manufacturing 710 a plurality of wearable electronic devices, distributing 720 one or more of the wearable devices of the plurality to each of multiple patients, collecting motion data 730 with the wearable electronic devices of the plurality, receiving 740 the wearable electronic devices of the plurality from the patients, physical processing 750 the wearable electronic devices, transferring 760 the motion data from the wearable electronic devices, processing 770 the motion data, and outputting 780 the assessed motion data to a prescriber, and may also include 790 repeating the operations 710 to 780 for another plurality of the wearable electronic devices for additional patients.

The manufacturing 710 of the plurality of the wearable electronic devices, such as the wearable electronic devices 100, is performed with a manufacturing system, such as the manufacturing system 610. The manufacturing 710 may be repeated for further pluralities of the wearable electronic devices by restoring the electronics 120 and/or the body 110 of the wearable electronic devices of an earlier plurality of the electronic devices (e.g., from previously worn wearable electronic devices).

The distributing 720 of one or more of the electronic devices of the plurality to each of one or more patients includes providing, to the patients, the wearable electronic devices as prescribed by a prescriber. The prescriber may be the distributor (e.g., a prescribing distributor) or may be another distributor (e.g., a non-prescribing distributor). The distributor associates the electronics devices provided to the patient with the patient (e.g., with identifiers as described previously). The distributor may also associate the wearable electronic devices with prescriber, thus the distributing 720 may further include receiving the prescriber information with the data system 630 described previously.

The collecting of the motion data 730 is performed with the wearable electronic devices. For example, depending on the configuration of the wearable electronic devices, the collecting of the motion data 730 may be performed with the wearable electronic devices according to the method 500, or any other suitable method.

The receiving 740 of the wearable electronic devices of the plurality is performed with a receiving system, such as the receiving system 620, of a processing facility, such as the processing facility 640. The receiving 740 includes receiving 740 the wearable electronic devices from the patients via parcel or other shipments of individual ones of the wearable electronic devices.

The physical processing 750 of the wearable electronic devices is performed, for example, with the receiving or another processing system. The physical processing 750 includes processing the wearable electronic devices to provide physical access to the electronics of the wearable electronic devices for connecting to the communications interface thereof for transferring the motion data therefrom and for restoring the electronics thereof for use in a wearable electronic device of a subsequent plurality of the wearable electronic devices. The physical processing 750, accordingly, includes one of removing the electronics from a band of the wearable electronic device, such as the body 110 (e.g., from the housing portion 112 thereof), or may include removing the removable cover, such as the removable segment 112c, from the band. The physical processing 750 may also include removing and/or discarding a power source, such as the power source 260, from the electronics. The physical processing 750 may include permanently deforming the band of the wearable electronic device, or the housing portion thereof, for example, to provide the physical access to the electronics.

The physical processing 750 may be omitted for wearable electronic devices that transfer data and/or power wirelessly.

The transferring 760 of the motion data includes connecting to the communications interfaces of the wearable electronic devices with a computing system, such as the data system 630, such as with a communications interface of a computing device thereof (e.g., the communications interface 632a of the computing device 632). The connection may be made physically (e.g., through conductive contacts) or wirelessly, depending on the configuration of the wearable electronic devices. The transferring 760 may also include providing a security key to the wearable electronic device, which may be required for retrieving or otherwise accessing the motion data of each of the wearable electronic devices.

The processing 770 of the motion data includes processing the motion data to assess activity of the patient. For example, the motion data may be processed to quantify activity according to time (e.g., steps per hour or per day, or other suitable quantification of activity). The processing 770 of the motion data is performed with the data system and a computing device thereof, such as the computing device that performed the transferring 760 or another computing device that received the motion data from such computing device. The processing of the motion data may also include associating the motion data with dates and times, for example, by backdating the time indicators (e.g., counter) associated with motion data based on a known (e.g., current) date and time. The processing 770 of the motion data may also include identifying motion data that corresponds the motion of the patient wearing the wearable electronic device as opposed to other motion thereof (e.g., during transport). The processing 770 may also include decrypting the motion data from each of the wearable electronic devices. The processing 770 may include generating a patient activity assessment report that includes a quantification of the patient activity.

The outputting 780 of the assessed motion data includes sending, providing access, or otherwise outputting the processed motion data (e.g., a quantification of the patient activity) to the prescriber. Information of the prescriber is received by the data system as part of the distributing 720 of the wearable electronic devices. The outputting 780 is performed with the data system, such as a computing device thereof, which may be the same or different computing devices from that or those performing the transferring 760 and the processing 770 of the motion data. The outputting 780 may be anonymized to the patient, for example, with neither the data system nor the wearable electronic device having received identified information of the patient. Rather, the prescriber may associate the assessed motion data with the patient. Or alternatively, the outputting 780 include providing the assessed motion data in association with the patient identifier.

The repeating 790 includes repeating the operations 710 to 780 for subsequent pluralities of the wearable electronic devices (e.g., second, third, fourth, and more). In some embodiments, the manufacturing 710 may include restoring the electronics of the wearable electronic devices, which are prepared during the physical processing 750 of the wearable electronic devices of an earlier plurality of the wearable electronic devices, which may include restoring the power source (e.g., replacing a battery) and restoring the data storage (e.g., by removing the motion data of a previous patient). The repeated manufacturing 710 includes resealing the electronics, such as in the electronics housing of an existing band (e.g., by providing a new removable seal, such as by applying new silicone or other seal), or by forming a new band around the electronics (e.g., molding the silicone of the new band around the electronics to encase the electronics in the electronics housing portion). The manufacturing 710 further includes, in the case of the power source being a metal air battery, preventing air from reaching battery, such as by sealing the wearable electronic device in an air-impermeable package or attaching a removable air-impermeable cover (e.g., sticker).

In addition to and/or consistent with the foregoing description, the following embodiments are contemplated:

1. A wearable electronic device comprising:
a motion sensing unit that senses motion and outputs motion data according thereto;
a data storage that receives and stores the motion data;
a communications interface for transferring the motion data from the data storage;
a power source for providing power to the motion sensing unit, the data storage, and the communications interface; and
an electronics housing configured to be worn by a patient and coupled to the motion sensing unit, the data storage, the communications interface, and the power source, wherein to one or more of transfer the motion data from the data storage or physically access the power source, the electronics housing must be permanently deformed.

2. The wearable electronic device according to Embodiment 1, comprising a band formed of a polymeric compound and coupleable to the patient.

3. The wearable electronic device according to Embodiment 2, wherein the polymeric compound is an elastomer and is formed monolithically around the motion sensing unit, the data storage, and the communications interface in a molding operation.

4. The wearable electronic device according to Embodiment 3, wherein the motion sensing unit, the data storage, and the communications interface are sealed with the electronics housing by the elastomer.

5. The wearable electronic device according to Embodiment 2, wherein the band comprises an elongated portion configured to couple the wearable electronic device to a wrist of the patient, wherein the elongated portion and the electronics housing are formed monolithically with the elastomer.

6. The wearable electronic device according to Embodiment 1, further comprising a power source that is a primary battery.

7. The wearable electronic device according to Embodiment 6, wherein to physically access the power source, the electronics housing must be permanently deformed.

8. The wearable electronic device according to Embodiment 7, wherein the motion sensing unit, the data storage, the communications interface, and the primary battery are sealed in the electronics housing that is formed of an elastomer.

9. The wearable electronic device according to Embodiment 6, wherein the motion sensing unit, the data storage, and the communications interface were previously used in another wearable electronic device and the power source was not used in the other wearable electronic device.

10. The wearable electronic device according to Embodiment 6, wherein the primary battery is a metal-air battery, wherein the wearable electronic device is provided one of sealed in air-impermeable packaging that prevent air from reaching the metal-air battery or with a removable air-impermeable cover that prevents air from reaching the metal-air battery.

11. The wearable electronic device according to Embodiment 1, wherein to transfer the motion data from the data storage, the electronics housing must be permanently deformed to physically access the communications interface.

12. A wearable electronic device for collecting motion data of a patient comprising:
a motion sensing unit having one or more sensors for sensing motion and outputting motion data according thereto;
a data storage for receiving and storing the motion data;
a communications interface for transferring the motion data from the data storage;
a controller for operating the motion sensing unit and the data storage; and
a power source;
wherein the wearable electronic device does not include any output device by which the patient can directly observe an output of the wearable electronic device, and does not include an input device by which the patient can provide an intentional input to the wearable electronic device.

13. The wearable electronic device according to Embodiment 12, wherein the wearable electronic device is configured to not provide any electronic output to and to not receive any electronic input from any electronic device of the patient.

14. The wearable electronic device according to Embodiment 13, wherein the wearable electronic device is configured to not provide any electronic output to and to not receive an electronic input from any electronic device of a prescriber of the wearable electronic device to the patient.

15. A method for assessing activity of multiple patients, comprising:
distributing one or more wearable electronic devices of a plurality of the wearable electronic devices to each of one or more patients to be worn thereby;
collecting, with each of the wearable electronic devices of the plurality, motion data of the patient while the wearable electronic device is being worn;
receiving, at a processing facility, each of the wearable electronic devices of the plurality from the patients; and
transferring, with a computer data system associated with the processing facility, the motion data from each of the wearable electronic devices of the plurality.

16. The method according to Embodiment 15, further comprising processing, with the computer data system, the motion data to assess activity of each of the patients.

17. The method according to Embodiment 16, further comprising outputting, with the computer data system, assessed activity data to a prescriber of each of the wearable electronic devices.

18. The method according to Embodiment 15, wherein the receiving includes receiving the wearable electronic devices of the plurality from the patients in shipments of individual ones of the wearable electronic devices of the plurality.

19. The method according to Embodiment 15, further comprising physically processing each of the wearable electronic devices of the plurality after the receiving by permanently deforming the wearable electronic device to provide access to a communications interface of the wearable electronic devices from which the motion data may be transferred from a data storage of the motion data.

20. The method according to Embodiment 19, wherein each of the wearable electronic devices includes electronics that include one or more motion sensors for sensing motion and outputting the motion data according thereto, the data storage for receiving and storing the motion data, and the communications interface, and the processing includes restoring the electronics of each of the wearable electronic devices for use in another wearable electronic device of another plurality of the wearable electronic devices.

21. The method according to Embodiment 20, wherein the electronics of each of the wearable electronics devices further includes a primary battery, and the restoring includes replacing the primary battery.

22. The method according to Embodiment 21, further comprising distributing one or more of the wearable electronic devices of the other plurality to one or more different patients.

23. The method according to Embodiment 15, further comprising, after transferring the motion data from each of the wearable electronic devices of the plurality, one of discarding all electronics of each of the wearable electronic devices or restoring a data storage of each of the wearable electronic devices by storing a new unique identifier in the data storage.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A wearable electronic device comprising:
a trigger for activating the wearable electronic device, the trigger being one of a physical trigger or an optical trigger;
a timer that provides time indicators;
a motion sensing unit that senses motion and outputs motion data according thereto; and
a data storage that receives and stores the motion data or other data derived therefrom in association with the time indicators;
wherein upon activating the wearable electronic device with the trigger, the timer begins providing the time indicators and the motion sensing unit begins operating to sense the motion; and
wherein the timer continues providing the time indicators until subsequent data transfer between the data storage and a computing device.

2. The wearable electronic device according to claim 1, further comprising a communications interface by which the motion data or the other data and the time indicators are transferred from the data storage to the computing device, a power source, and a housing portion in which the timer, the motion sensing unit, the data storage, the communications interface, and the power source are positioned;
wherein physical access to the communications interface requires permanent deformation of the housing portion;
wherein the trigger activates the wearable electronic device by causing the power source to begin providing power to the motion sensing unit; and
wherein the trigger is the physical trigger and is configured as an electrically insulative member that is removable from between electrical contacts to close a circuit therebetween.

3. The wearable electronic device according to claim 1, wherein the trigger is the optical trigger configured and is configured as a light sensor that activates the wearable electronic device after sensing environmental light.

4. The wearable electronic device according to claim 1, wherein the trigger is the physical trigger and is configured as an electrically insulative member that is removable from between electrical contacts to close a circuit therebetween.

5. The wearable electronic device according to claim 1, further comprising a communications interface by which the motion data or the other data and the time indicators are transferred from the data storage to the computing device.

6. The wearable electronic device according to claim 5, further comprising a housing portion in which the timer, the motion sensing unit, the data storage, and the communications interface are positioned.

7. The wearable electronic device according to claim 6, wherein physical access to the communications interface requires permanent deformation of the housing portion.

8. The wearable electronic device according to claim 7, wherein the housing portion includes a removable segment that requires permanent deformation of the housing portion to provide the physical access to the communications interface.

9. The wearable electronic device according to claim 6, further comprising a removable segment that is removable from the housing portion to provide physical access to the communications interface, the removable segment being a separate member that is coupled to the housing portion.

10. The wearable electronic device according to claim 1, further comprising a power source, wherein the trigger activates the wearable electronic device by causing the power source to begin providing power to the motion sensing unit.

11. A wearable electronic device comprising:
a trigger for activating the wearable electronic device;
a timer that provides time indicators;
a motion sensing unit that senses motion and outputs motion data according thereto; and
a data storage that receives and stores the motion data or other data derived therefrom in association with the time indicators.

12. The wearable electronic device according to claim 11, further comprising a power source, wherein the trigger activates the wearable electronic device by causing the power source to begin providing power to the motion sensing unit; and
further comprising a proximity sensor, wherein upon activating the wearable electronic device with the trigger, the proximity sensor begins operating to sense a patient wearing the wearable electronic device;
wherein after sensing the patient wearing the wearable electronic device, the motion sensing unit begins sensing the motion; and
wherein the data storage stores the other data that is the root mean square of motion data from each of three axes of the motion sensing unit.

13. The wearable electronic device according to claim 11, wherein upon activating the wearable electronic device with the trigger, the timer continues providing the time indicators until subsequent data transfer between the data storage and a computing device.

14. The wearable electronic device according to claim 11, further comprising a proximity sensor, wherein upon activating the wearable electronic device with the trigger, the proximity sensor begins operating to sense a patient wearing the wearable electronic device; and
wherein after sensing the patient wearing the wearable electronic device, the motion sensing unit begins sensing the motion.

15. The wearable electronic device according to claim 11, further comprising a power source, wherein the trigger activates the wearable electronic device by causing the power source to begin providing power to the motion sensing unit.

16. The wearable electronic device according to claim 11, wherein the data storage stores the other data that is the root mean square of motion data from each of three axes of the motion sensing unit.

17. A method for assessing activity of multiple patients comprising:
manufacturing wearable electronic devices;
distributing one or more of the wearable electronic devices to each of multiple patients;
collecting motion data for each patient with the wearable electronic device distributed thereto; and
transferring the motion data from the wearable electronic devices at a processing facility.

18. The method according to claim 17, further comprising:
receiving, at the processing facility, the wearable electronic devices for each of the patients;
physically processing, at the processing facility, each of the wearable electronic devices by connecting to a communications interface thereof to transfer the motion data therefrom to a computing device; and
processing, at the processing facility, the motion data transferred from the wearable electronic devices to form assessments of patient activity for each of the patients, and send, for each of the patients, the assessments of patient activity to a prescriber that prescribed the wearable electronic device for the patient.

19. The method according to claim 17, further comprising:
receiving, at the processing facility, the wearable electronic devices for each of the patients; and
physically processing, at the processing facility, each of the wearable electronic devices by connecting to a communications interface thereof to transfer the motion data therefrom to a computing device.

20. The method according to claim 17, further comprising processing, at the processing facility, the motion data transferred from the wearable electronic devices to form assessments of patient activity for each of the patients, and send, for each of the patients, the assessments of patient activity to a prescriber that prescribed the wearable electronic device for the patient.
